(19) United States
(12) Patent Application Publication
(10) Pub. No.: US 2023/0340611 A1
(43) Pub. Date: Oct. 26, 2023

(54) Biomarkers For Immune Checkpoint Inhibitors Treatment

(71) Applicant: Novigenix SA, Epalinges (CH)

(72) Inventors: Sahar HOSSEINIAN EHRENSBERGER, Epalinges (CH); Victoria WOSIKA, Epalinges (CH); Laura CIARLONI, Epalinges (CH); Sylvain MONNIER-BENOIT, Epalinges (CH)

(21) Appl. No.: 18/025,146

(22) PCT Filed: Sep. 16, 2021
(86) PCT No.: PCT/EP2021/075488

§ 371 Date: Mar. 7, 2023

(30) Foreign Application Priority Data
Sep. 16, 2020 (EP) 20196385.7

(51) Int. Cl. C12Q 1/6886; G16B 25/10

(52) U.S. Cl. CPC C12Q 1/6886; G16B 25/10; C12Q 2600/106; C12Q 2600/158

(57) ABSTRACT

The present invention relates to methods for determining or predicting if a patient having a predetermined disease, for example cancer, in particular metastatic urothelial cancer, is responsive, or will respond to a treatment based on immune checkpoint inhibitor. The present invention also relates to computer-implemented methods for implementing said methods.

### FIELD OF THE INVENTION

The present invention relates to methods for determining or predicting if a patient having a predetermined disease, for example cancer, in particular metastatic urothelial cancer, is responsive, or will respond to a treatment based on immune checkpoint inhibitor. The present invention also relates to computer-implemented methods for implementing said methods.

### BACKGROUND OF THE INVENTION

Immune checkpoint inhibitors (ICIs) have become an integral part of therapy for patients with metastatic urothelial cancer (mUC). Since a few years, ICIs targeting the programmed cell death protein 1 (PD-1)/ programmed cell death ligand 1 (PD-L1) axis are used to treat cisplatin-ineligible patients with a PD-L1 positive tumor as well as patients that have progressed on first-line platinum-based chemotherapy. Additionally, maintenance therapy with PD-L1 inhibitor avelumab was recently approved for the treatment of patients who achieved a response or stable disease with first-line chemotherapy. Although anti-PD-(L)1 prolongs median overall survival (OS), only a minority of patients benefit from it [1]. In a phase III clinical trial, second-line treatment with PD-1 inhibitor pembrolizumab induced responses in 21.1% and disease control in 38.5% of mUC patients [1].

Application of biomarkers would limit the use of PD-(L)1 inhibitors in patients that do not benefit from it, thereby preventing immune-related toxicity and enabling the rapid introduction of other, potentially more effective therapies. Several promising treatment strategies have emerged and are either in late-stage clinical trials or already approved by the Food and Drug Administration for the treatment of mUC. Recently approved drugs include enfortumab vedotin and erdafitinib. Additionally, dual checkpoint inhibition is currently being studied in various disease settings and might be beneficial in some patients that do not benefit from anti-PD-(L)1 monotherapy.

So far, efforts have focused on the identification of predictive biomarkers that can be obtained prior to treatment initiation. Although tumor mutational burden, PD-L1 expression and CD8⁺ T cell infiltration at baseline appear to enrich for response to ICIs [2-6], these biomarkers are not accurate enough to be used as stand-alone biomarkers. Early response biomarkers may also have clinical utility but have been underexplored. In current practice, the first radiological response evaluation is usually not performed until after 12 weeks of ICI therapy and is sometimes equivocal. Clinically stable patients with suspected progression may continue treatment after the first scan according to iRECIST to avert treatment discontinuation in patients with delayed responses or pseudo-progression. Early blood-based response biomarkers may provide a reliable way to determine whether ICIs are effective before imaging is available and can be particularly useful for those with equivocal imaging.

Translational studies in patients with various tumor types indicate that clinical benefit to ICIs is accompanied by systemic immunological changes during the first weeks of treatment. In patients with melanoma or lung cancer, decreases in IL-6 and IL-8 during the first weeks of therapy have been associated with improved outcome to ICIs [7, 8]. Additionally, a study in patients with melanoma or Merkle cell carcinoma demonstrated that a high frequency of circulating PD-1⁺ TIGIT⁺ CD8⁺ T cells after 1 month of anti-PD-1 was associated with an increased response rate and longer OS [9]. Furthermore, studies in lung cancer and melanoma have described an association between T cell proliferation and response to therapy [10, 11]. However, data on ICI-induced changes in peripheral blood of mUC patients are lacking.

Biomarkers that can both predict clinical outcome and help determining a patient’s responsiveness to immune checkpoint blockade therapy or treatment (ICBT) are urgently needed.

### SUMMARY OF THE INVENTION

The present invention provides a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and,
at least one gene selected among those listed in Table 1,
   wherein the at least one ECM gene cluster comprises COL14A1 and the at least one gene of Table 1 comprises MORN4A, and
   wherein differential transcription and/or expression and/or activity level of the gene panel, in the biological sample, relative to the level of corresponding transcription and/or expression and/or activity level of the gene panel determined previously, is predictive of the patient’s response to said treatment.

Further provided is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and
wherein differential transcription and/or expression and/or activity level of the gene panel, in the biological sample, relative to the level of corresponding transcription and/or expression and/or activity level of the gene panel determined previously, is predictive of the patient’s response to said treatment.

Further provided is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among those listed in Table 1, and
wherein differential transcription and/or expression and/or activity level of the gene panel, in the biological sample, relative to the level of corresponding transcription and/or expression and/or activity level of the gene panel determined previously, is predictive of the patient’s response to said treatment.

Further provided is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the cAMP cluster (Table 3),
wherein differential transcription and/or expression and/or activity level of the gene panel, in the biological sample, relative to the level of corresponding transcription and/or expression and/or activity level of the gene panel determined previously, is predictive of the patient’s response to said treatment.

Further provided is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the DNA replication cluster of Table 4,
wherein differential transcription and/or expression and/or activity level of the gene panel, in the biological sample, relative to the level of corresponding transcription and/or expression and/or activity level of the gene panel determined previously, is predicting that the patient is responsive to said treatment.

Further provided is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Interferon cluster genes (Table 5),
wherein differential transcription and/or expression and/or activity level of the gene panel, in the biological sample, relative to the level of corresponding transcription and/or expressionand/or activity level of the gene panel determined previously, is predicting that the patient is responsive to said treatment.

Further provided is a method for determining if a patient having a predetermined disease is responsive to a treatment based on immune checkpoint blockade therapy or treatment (ICBT), said method comprising detecting in a biological sample obtained from said patient the level of transcription and/or expression and/or activity of a gene panel comprising:
at least one gene selected among the DNA replication cluster (Table 4) and,
at least one gene selected among the gene interferon cluster (Table 5),
   wherein the at least one DNA replication gene cluster comprises PLK4 and the at least one interferon cluster gene comprises PDCD1, and
   wherein differential transcription and/or expression and/or activity level of the gene panel, in the biological sample, relative to the level of corresponding transcription and/or expression and/or activity level of the gene panel determined previously, is indicative of whether the patient responds or not to said treatment.

Further provided is a computer-implemented method for implementing a method for predicting if a patient having a predetermined disease will respond to a treatment based on immune checkpoint blockade therapy or treatment (ICBT) of the invention, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined previously, whereby differential transcription and/or expression and/or activity level of the gene panel, in the biological sample, relative to the level of corresponding transcription and/or expression and/or activity level of the gene panel determined previously, is predictive of the patient’s response to said treatment.

Further provided is a computer-implemented method for implementing a method for determining if a patient having a predetermined disease is responsive to a treatment based on immune checkpoint blockade therapy or treatment (ICBT) of the invention, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined in a control biological sample, whereby wherein differential transcription and/or expression and/or activity level of the gene panel, in the biological sample of the patient, relative to the level of corresponding transcription and/or expression and/or activity level of the gene panel determined previously, is indicative of whether the patient will respond or not to said treatment.

Further provided is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the list of Table 6,
wherein differential transcription and/or expression and/or activity level of the gene panel, in the biological sample, relative to the level of corresponding transcription and/or expression and/or activity level of the gene panel determined previously, is predicting that the patient is responsive to said treatment.

Further provided is the use of a gene panel comprising at least one gene selected among the Extra Cellular Matrix (ECM) cluster (Table 2) and, at least one gene selected among those listed in Table 1, wherein the at least one ECM gene cluster comprises COL14A1 and the at least one gene of Table 1 comprises MORN4A, for predicting if a patient having a predetermined disease will respond to a treatment based on immune checkpoint blockade therapy or treatment (ICBT).

Further provided is the use of a gene panel comprising at least one gene selected among the DNA replication cluster (Table 4) and, at least one gene selected among the gene interferon cluster (Table 5), wherein the at least one DNA replication gene cluster comprises PLK4 and the at least one interferon cluster gene comprises PDCD1, for determining if a patient having a predetermined disease is responsive to a treatment based on immune checkpoint blockade therapy or treatment (ICBT).

Further provided is the use of a gene panel for predicting if a patient having a predetermined disease will respond to a treatment based on immune checkpoint blockade therapy or treatment (ICBT), comprising:
at least one gene selected among the Extra Cellular Matrix (ECM) cluster (Table 2) and/or
at least one gene selected among those listed in Table 1, and/or
at least one gene selected among the cAMP cluster (Table 3), and/or
at least one gene selected among the DNA replication cluster of Table 4.

Further provided is the use of a gene panel for determining if a patient having a predetermined disease is responsive to a treatment based on immune checkpoint blockade therapy or treatment (ICBT), comprising:
at least one gene selected among the DNA replication cluster of Table 4, and/or
at least one gene selected among the Interferon cluster genes (Table 5), and/or
at least one gene selected among those listed in Table 6.

Further provided is a kit for performing a method according to the invention, said kit comprising
a) means and/or reagents for determining the level of transcription and/or expression and/or activity of said gene panel in a biological sample from said patient, and
b) instructions for use.

Further provided is a method of treatment of a cancer or an autoimmune disease, comprising
i) detecting in a biological sample obtained from said patient the level of transcription and/or expression and/or activity of a gene panel of any one of tables 1, 2, and/or 3,
ii) and treating the patient based upon whether a differential transcription and/or expression and/or activity level of said gene panel, in the biological sample, relative to the level of corresponding transcription and/or expression and/or activity level of the gene panel determined previously, is predictive of the patient’s response to said treatment.

Further provided is a method of treatment of a cancer or an autoimmune disease, comprising
i) detecting in a biological sample obtained from said patient the level of transcription and/or expression and/or activity of a gene panel of any one of tables 4, 5, and/or 6,
ii) and treating the patient based upon whether a differential transcription and/or expression and/or activity level of said gene panel, in the biological sample, relative to the level of corresponding transcription and/or expression and/or activity level of the gene panel determined previously, is predicting that the patient is responsive to said treatment.

### DESCRIPTION OF THE FIGURES

FIG. 1 - Volcano plot of Differentially expressed genes (DEGs) between responders and non-responders at baseline. The bold line indicates the significance threshold (padj = 0.01) and dashed line a padj = 0.05.

FIGS. 2 - ROC curve depicting the performance of the classifiers “subset 6” (a) and “subset 3” (b) for predicting response to therapy at baseline.

FIGS. 3 - Kaplan-Meier curves. Progression-free survival in patients classified as high and low score by the 105-gene model (A), by the ECM gene model (B), by the cAMP model (C). The stratum “0” (black line) refer to non-Responders (CB-) and the stratum “1” (grey line) to Responders (CB+). Time is expressed in days.

FIG. 4 -Volcano Plot of differentially expressed genes (DEGs) between baseline and on-treatment samples in patients with clinical benefit. The dashed line indicates the significance threshold (padj = 0.05). In patients with clinical benefit, 51 DEGs were identified.

FIGS. 5 - Kaplan-Meier curves. (A) Progression-free survival in patients classified as Responder or Non-responder by the classifier including 5 DNA replication genes ( DLGAP5, TOP2A, CDCA2, E2F8 and SMC1A). (C) Progression-free survival in patients classified as Responder or Non-responder by the classifier including 5 DNA replication and 1 IFN gene ( DLGAP5, TOP2A, CDCA2, E2F8, SMC1A and PDCD1). (B) Progression-free survival in patients with versus without an above-median increase in PDCD1 gene expression. Light grey line: Non-responder; Dark grey line: Responder

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc.

As used herein the terms ″subject″/″patient″, are well-recognized in the art, and are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human patient having a predetermined disease, more preferably the predetermined disease is a cancer or an autoimmune disease.

In one aspect, the predetermined disease is a cancer, whether solid or liquid, selected from the non-limiting group comprising urothelial cancer, urinary bladder cancer, lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, hepatic cancer, brain cancer and skin cancer, or a combination of one or more thereof. Preferably, the urinary bladder cancer is urothelial cancer, more preferably metastatic urothelial cancer (mUC).

As used herein, an “autoimmune disease” represents a member of a family of at least 80 diseases that share a common pathogenesis: an improper activation of the immune system attacking the body’s own organs. In one aspect, the autoimmune disease is selected from the group comprising rheumatoid arthritis, systemic lupus erythematosus, multiple sclerosis, type-1 diabetes, autoimmune hepatitis, inflammatory bowel disease, and myocarditis. PD-1, PD-L1 and/or CTLA-4 signaling has/have been shown to be involved in the pathogenesis of many autoimmune diseases including those listed above.

In one aspect, the treatment of the invention is based on immune checkpoint blockade therapy or treatment (ICBT). Preferably, said treatment based on ICBT is selected among the group comprising a PD-1 inhibitor, a PD-L1 inhibitor and a CTLA-4 inhibitor, or combination of one or more thereof (e.g. PD-1/PD-L1 inhibitor or CTLA-4/ PD-1 inhibitor).

In a preferred aspect, the treatment based on ICBT comprises treatment with monoclonal antibodies (mAbs) specific to PD-1, PD-L1 or CTLA-4, or a combination of one or more thereof (see e.g. Rotte, A. Combination of CTLA-4 and PD-1 blockers for treatment of cancer. J Exp Clin Cancer Res 38, 255 (2019); Twomey, J.D., Zhang, B. Cancer Immunotherapy Update: FDA-Approved Checkpoint Inhibitors and Companion Diagnostics. AAPS J 23, 39 (2021)).

Non-limiting examples of mAbs specific to PD-1 comprise Nivolumab, Pembrolizumab, and Cemiplimab.

Non- limiting examples of mAbs specific to PDL-1 comprise Atezolizumab, Avelumab, and Durvalumab.

As defined herein, patients were classified as responder (CB+) or non-responder (CB-). Patients were considered to have clinical benefit (responder) if they had a radiological and clinical progression-free survival (PFS) of at least 6 months.

As discussed herein, the level of transcription and/or expression and/or activity of a gene panel may be expressed as a score. The score may be calculated as the mean, or the median, or the ratio or the sum, or the weighted mean, median or the sum, the ratio of the expression levels of the genes composing the panel in control samples and disease samples.

Alternatively, the score may be calculated as the first component or multiple components of Principal Component Analysis (PCA), or Neural Network dimensional embeddings or any Dimensionality reduction method.

Also it can be calculated as a probability of a prediction model using generalized linear models, or Lasso and Elastic-Net Regularized Generalized Linear Models, Sparse partial least squares regression, or nearest-centroid classification, or nearest shrunken centroid, or neural networks or random forest, or support vector machine, or naïve bayes, or K-means.

A “pre-defined score” refers to a mathematical formula that has been determined by fitting a predictive model at training phase on the training data set for instance by logistic regression. The fitted model will be used to calculate the score or predicting the likelihood of being responsive to the therapy for each new patient. The bootstrap method or the cross-validation method with a ROC analysis can estimate the performances of the fitted model in each mathematical method.

As used herein, a biological sample may include a body fluid or body cell or tissue and is selected from the group comprising whole blood, serum, plasma, semen, saliva, tears, urine, fecal material, sweat, buccal smears, skin, tumor tissue, cancer cells, or a combination of one or more of thereof. More preferably, the biological sample is selected from the group comprising whole blood sample, tumor tissue sample and cancer cell sample.

The inventors conducted a study aimed at the identification of predictive and early markers of response to ICBT in patients with cancer, in particular metastatic urothelial cancer. By performing a comprehensive, unbiased whole blood transcriptome analysis, they surprisingly revealed that
one or more genes listed in tables 1, 2 and/or 3, are up or down regulated thus predicting if a patient will respond to a treatment based on ICBT, whereas
one or more genes listed in tables 4, 5 and/or 6, are up or down regulated thus determining if a patient is responsive to a treatment based on ICBT.

In one aspect, the invention relates to a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and,
at least one gene selected among those listed in Table 1,
   wherein the at least one ECM gene cluster comprises COL14A1 and the at least one gene of Table 1 comprises MORN4A, and
   wherein differential transcription and/or expression and/or activity level of the gene panel, in the biological sample, relative to the level of corresponding transcription and/or expression and/or activity level of the gene panel determined previously, is predictive of the patient’s response to said treatment.

In one aspect, the at least one gene selected among those listed in Table 1 is further selected from the group comprising BNIPL, CCDC40, and DHRS2, or a combination of two or more thereof, e.g. BNIPL and CCDC40, BNIPL and DHRS2, or DHRS2 and CCDC40. In one aspect, the least one gene selected among those listed in Table 1 consists of the combination of MORN4A, BNIPL, CCDC40, and DHRS2.

Preferably, the gene panel further comprises at least two genes selected among those listed in Table 1, preferably a combination of three or more thereof, preferably a combination of five or more thereof, preferably a combination of ten or more thereof, preferably a combination of twenty or more thereof, preferably a combination of thirty or more thereof, preferably a combination of forty or more thereof, preferably a combination of fifty or more thereof, preferably a combination of sixty or more thereof, preferably a combination of seventy or more thereof, preferably a combination of eighty or more thereof, or more preferably a combination of ninety-two thereof.

In one aspect, the least one gene selected among the Extra Cellular Matrix (ECM) cluster (Table 2) is further selected from the group comprising DOCK1 and ADAMTS2, or a combination thereof.

Preferably, the gene panel further comprises at least two genes selected among those listed in Table 2, preferably a combination of three or more thereof, preferably a combination of five or more thereof, preferably a combination of ten or more thereof, preferably a combination of twenty or more thereof, preferably a combination of thirty or more thereof, or more preferably a combination of all the genes listed in Table 2.

In a further aspect, the gene panel further comprises at least one gene selected among the cAMP cluster (Table 3). Preferably, said at the least one gene selected among the cAMP cluster (Table 3) is selected from the group comprising PDE10A, CASR, and KCNJ6, or a combination of two or more thereof, e.g. PDE10A and CASR, PDE10A and KCNJ6, or CASR, and KCNJ6. In one aspect, the least one gene selected among the cAMP cluster (Table 3) consists of the combination of PDE10A, CASR, and KCNJ6.

Preferably, the gene panel further comprises at least one gene selected among those listed in Table 3, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, or preferably a combination of twelve or more thereof, preferably a combination of thirteen or more thereof, preferably a combination of fourteen or more thereof, or preferably a combination of all the genes listed in Table 3.

In a preferred aspect of the invention, the treatment is based on immune checkpoint blockade therapy or treatment (ICBT) and is selected among the group comprising a PD-1 inhibitor, a PD-L1 inhibitor and a CTLA-4 inhibitor, or combination of one or more thereof as discussed herein.

Usually, a differential transcription and/or expression and/or activity level of the gene panel corresponds to a differential expression of the transcripts (e.g. RNA or mRNA) of the genes of the panel. This differential transcription and/or expression and/or activity level of the gene panel can correspond to a downregulated or upregulated expression of said genes. Preferably, the differential transcription and/or expression and/or activity level of the gene panel corresponds to a downregulated expression of said genes.

Preferably, the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously.

Usually, the level of corresponding transcription and/or expression and/or activity level of the gene panel determined previously has been determined in a biological sample of the patient before starting the ICBT (i.e. control sample or baseline). Preferably, the determination has been done about at least 1 month before, about at least 1 week before, about at least one day before, about at least 1 hour, about at least 1 minute before starting the treatment. Alternatively, the biological sample has been collected before starting the treatment, but the determination is done after starting the treatment.

More preferably, the detection is, has been, or will be performed in a biological sample obtained from said patient having a predetermined disease.

Various techniques for determining differential transcription and/or expression and/or activity of a gene panel are known in the art.

For example, one may calculate differential expression of one gene in a test sample by, e.g. calculating the ratio (fold change) between the expression level of the gene in the test sample and the expression level of the gene in the reference sample or group of samples, or reference value.

Expression level can be measured as transcripts per million (TPM) by RNA seq, as Threshold cycles (Ct) by PCR, as probe fluorescence intensity by microarray, etc....

Determining transcriptional changes in a group of samples for all the transcriptome it usually done using computational methods to determine differential gene expression in a full RNAseq dataset (e.g. 15000 genes). Different commonly used methods are, e.g., selected among the following software packages (open source): edgeR, DESeq2, limma, Cuffdiff, PoissonSeq, baySeq, etc... Preferably, DESeq2 is used (Love, M.I., Huber, W. & Anders, S. Moderated estimation of fold change and dispersion for RNA-seq data with DESeq2. Genome Biol 15, 550 (2014)).

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker in the absence of treatment or after starting the treatment. These values or ranges can be obtained from a single patient or alternatively from a group of patients.

The transcripts of the genes of the invention can be detected and, alternatively, quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, mass spectrometry, and any RNA sequencing-based methods known in the art (such as e.g. whole transcriptome RNA seq, targeted RNA seq, single cell RNA seq, total RNA sequencing, mRNA sequencing, whole transcript RNA sequencing, 3′ RNA sequencing, long RNA sequencing, direct RNA sequencing).

It is understood that the expression level of the genes (e.g. biomarkers) in a sample can be determined by any suitable method known in the art. Measurement of the level of a gene can be direct or indirect. For example, the abundance levels of RNAs can be directly quantitated. Alternatively, the amount of a gene (biomarker) can be determined indirectly by measuring abundance levels of cDNAs, amplified RNAs or DNAs, or by measuring quantities or activities of RNAs, or other molecules that are indicative of the expression level of the gene (such as, e.g proteins). Preferably, the amount of a gene (biomarker) is determined indirectly by measuring abundance levels of cDNAs.

In one aspect, microarrays are used to measure the levels of genes (biomarkers). An advantage of microarray analysis is that the expression of each of the genes can be measured simultaneously, and microarrays can be specifically designed to provide a diagnostic expression profile for a particular disease or condition (e.g., a cancer).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one aspect, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one aspect, the microarray comprises a support or surface with an ordered array of binding (e.g., hybridization) sites or “probes” each representing one of the genes described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm2; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, RNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some aspects, the probes of the array consist of nucleotide sequences of 10 to 1,000 nucleotides. In aspect aspect, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other aspects, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism’s genome. In another aspect, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990). It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some aspects, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., U.S. Pat. No. 5,539,083).

Probes are preferably selected using an algorithm that takes into account binding energies, base composition, sequence complexity, cross-hybridization binding energies, and secondary structure.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one aspect, positive controls are synthesized along the perimeter of the array. In another aspect, positive controls are synthesized in diagonal stripes across the array. In still another aspect, the reverse complement for each probe is synthesized next to the position of the probe to serve as a negative control. In yet another aspect, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA A second method for making microarrays produces high-density oligonucleotide arrays. Techniques are known for producing arrays containing thousands of oligonucleotides complementary to defined sequences, at defined locations on a surface using photolithographic techniques for synthesis in situ (see, U.S. Pat. Nos. 5,578,832; 5,556,752; and 5,510,270) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189;. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm2. The polynucleotide probes are attached to the support covalently at either the 3′ or the 5′ end of the polynucleotide.

Biomarker which may be measured by microarray analysis can be expressed RNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one aspect, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., U.S. Pat. No. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnC12, to generate fragments of RNA.

In one aspect, total RNA, mRNAs, or nucleic acids derived therefrom (such as cDNA), are isolated from a sample taken from a patient having a predetermined disease. Biomarker that are poorly expressed in particular cells may be enriched using normalization techniques known in the art.

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001) . Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one aspect, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer-controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor mRNA abundance levels at a large number of sites simultaneously.

As discussed above, many RNA sequencing-based methods are available. Non-limiting examples comprise, e.g. whole transcriptome RNA seq, targeted RNA seq, single cell RNA seq, total RNA sequencing, mRNA sequencing, whole transcript RNA sequencing, 3′ RNA sequencing, long RNA sequencing, direct RNA sequencing. Each of these sequencing technologies have their own way of preparing samples prior to the actual sequencing step. Depending on the sequencing technology used, amplification steps may be omitted.

In case the patient having a predetermined disease (e.g. cancer) is predicted to respond to said treatment, the treatment is started. Usually, the patient is predicted to respond when the level of corresponding transcription and/or expression and/or activity level of the gene panel is downregulated.

In case the patient having a predetermined disease, (e.g. cancer) is predicted not to respond to said treatment, the method further comprises a step of adapting the treatment. Usually, the patient is predicted not to respond when the level of corresponding transcription and/or expression and/or activity level of the gene panel is upregulated or not significantly different to the level of the gene panel determined previously.

In one aspect, the step of adapting the treatment comprises not administering the envisioned treatment or inhibitor, switching to another treatment or inhibitor, and/or adapting the dose of the treatment or inhibitor.

In another related aspect, the invention relates to a method for determining if a patient having a predetermined disease is responsive to a treatment based on immune checkpoint blockade therapy or treatment (ICBT), said method comprising detecting in a biological sample obtained from said patient the level of transcription and/or expression and/or activity of a gene panel comprising:
at least one gene selected among the DNA replication cluster (Table 4) and,
at least one gene selected among the gene interferon cluster (Table 5),
   wherein the at least one DNA replication gene cluster comprises PLK4 and the at least one interferon cluster gene comprises PDCD1, and
   wherein differential transcription and/or expression and/or activity level of the gene panel, in the biological sample, relative to the level of corresponding transcription and/or expression and/or activity level of the gene panel determined previously, is indicative of whether the patient responds or not to said treatment.

In one aspect, the least one gene selected among the DNA replication cluster (Table 4) is further selected from the group further comprising CENPE, CDCA2, E2F8, TOP2A, DLGAP5, SGOL2, NOTCH3, CCNB2, ASPM, SMC1A and MCM10, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, or preferably a combination of all the genes listed in Table 4.

In one aspect, the least one gene selected among the gene interferon cluster is selected from the group further comprising CLC, NMI, FCGR1A, FCGR1B, BCL2L14, IFITM3, STAT1, GBP2, C5, IFIT5, IFI35, TRIM22, IL10, and IDO1, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of twelve or more thereof, preferably a combination of thirteen or more thereof, or preferably a combination of all the genes listed in Table 5.

In one aspect, the gene panel further comprises at least one gene selected among those listed in Table 6, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of fifteen or more thereof, preferably a combination of twenty or more thereof, preferably a combination of twenty five or more thereof, preferably a combination of thirty or more thereof, preferably a combination of thirty five or more thereof, preferably a combination of forty or more thereof, or preferably a combination of all the genes listed in Table 6.

In a preferred aspect of the invention, the treatment is based on immune checkpoint blockade therapy or treatment (ICBT) and is selected among the group comprising a PD-1 inhibitor, a PD-L1 inhibitor and a CTLA-4 inhibitor, or combination of one or more thereof as discussed herein.

In one aspect, the differential transcription and/or expression and/or activity level of the gene panel corresponds to a differential expression of the transcripts of the genes of the panel when compared to a control biological sample (i.e. of the same patient having started the treatment).

Preferably, the determination of the differential transcription and/or expression and/or activity level of the gene panel is performed about at least 1 week after, about at least one month after, about at least two months, etc... after starting the treatment.

This differential transcription and/or expression and/or activity level of the gene panel can correspond to a downregulated or upregulated expression of said genes. Preferably, the differential transcription and/or expression and/or activity level of the gene panel corresponds to a downregulated expression of said genes.

Preferably, the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously.

Preferably also, the upregulated differential transcription and/or expression and/or activity of said gene panel corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously.

In case the patient having a predetermined disease (e.g. cancer) is responsive to said treatment, the treatment is continued. In one aspect, the patient is determined as responsive when the level of corresponding transcription and/or expression and/or activity level of the gene panel is upregulated (log2FC >0) for genes selected from Tables 4, 5, or for genes of Table 6 that display a log2FC >0 as identified comparing ICBT responders (Res), or complete responders (ComRes) before and during therapy.

In one aspect, the patient is determined as responsive when the level of corresponding transcription and/or expression and/or activity level of the gene panel is downregulated for the genes of Table 6 displaying a log2FC < 0 as identified comparing ICBT responders (Res), or complete responders (ComRes) before and during therapy.

In case the patient having a predetermined disease (e.g. cancer) is not responsive to said treatment, the method further comprises a step of adapting the treatment. Usually, the patient is determined as not responsive when the level of corresponding transcription and/or expression and/or activity level of the gene panel is dowregulated for genes selected from Tables 4 and 5, or for genes of Table 6 that displayed a log2FC >0 as identified comparing ICBT responders (Res), or complete responders (ComRes) before and during therapy.

In one aspect, the patient is determined as not responsive when the level of corresponding transcription and/or expression and/or activity level of the gene panel is upregulated for the genes of Table 6 displaying a log2FC < 0 as identified comparing ICBT responders (Res), or complete responders (ComRes) before and during therapy.

In one aspect, the step of adapting the treatment comprises changing the treatment for another treatment or inhibitor and/or adapting the dose of the inhibitor.

As for the method of predicting described herein, the level of corresponding transcription and/or expression and/or activity level of the gene panel determined previously has been determined before starting the ICBT.

Also encompassed is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the DNA replication cluster of Table 4,
wherein differential transcription and/or expression and/or activity level of the gene panel, in the biological sample, relative to the level of corresponding transcription and/or expression and/or activity level of the gene panel determined previously, is predicting that the patient is responsive to said treatment.

In a preferred aspect, the least one gene selected among the DNA replication cluster (Table 4) is selected from the group further comprising PLK4, CENPE, CDCA2, E2F8, TOP2A, DLGAP5, SGOL2, NOTCH3, CCNB2, ASPM, SMC1A and MCM10, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of all the genes listed in Table 4.

In one aspect, the patient is determined as responsive when the level of corresponding transcription and/or expression and/or activity level of the gene panel is upregulated for genes selected from Table 4.

In case the patient having a predetermined disease (e.g. cancer) is responsive to said treatment, the treatment is continued.

Also encompassed is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Interferon cluster genes (Table 5),
wherein differential transcription and/or expression and/or activity level of the gene panel, in the biological sample, relative to the level of corresponding transcription and/or expression and/or activity level of the gene panel determined previously, is predicting that the patient is responsive to said treatment.

In a preferred aspect, the least one gene selected among the gene interferon cluster is selected from the group comprising PDCD1, CLC, NMI, FCGR1A, FCGR1B, BCL2L14, IFITM3, STAT1, GBP2, C5, IFIT5, IFI35, TRIM22, IL10, and IDO1, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of twelve or more thereof, preferably a combination of thirteen or more thereof, preferably a combination of fourteen or more thereof, or preferably a combination of all the genes listed in Table 5.

In one aspect, the patient is determined as responsive when the level of corresponding transcription and/or expression and/or activity level of the gene panel is upregulated for genes selected from Table 5.

In case the patient having a predetermined disease (e.g. cancer) is responsive to said treatment, the treatment is continued.

Also encompassed is a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the list of Table 6,
wherein differential transcription and/or expression and/or activity level of the gene panel, in the biological sample, relative to the level of corresponding transcription and/or expressionand/or activity level of the gene panel determined previously, is predicting that the patient is responsive to said treatment.

In a preferred aspect, the at least one gene selected among the list of Table 6, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of fifteen or more thereof, preferably a combination of twenty or more thereof, preferably a combination of twenty five or more thereof, preferably a combination of thirty or more thereof, preferably a combination of thirty five or more thereof, preferably a combination of forty or more thereof, or preferably a combination of all the genes listed in Table 6.

In one aspect, the patient is determined as responsive when the level of corresponding transcription and/or expression and/or activity level of the gene panel is upregulated for genes of Table 6 that displayed a log2FC >0 as identified comparing ICBT responders (Res), or complete responders (ComRes) before and during therapy.

In one aspect, the patient is determined as not responsive when the level of corresponding transcription and/or expression and/or activity level of the gene panel is upregulated for the genes of Table 6 displaying a log2FC < 0 as identified comparing ICBT responders (Res), or complete responders (ComRes) before and during therapy.

In case the patient having a predetermined disease (e.g. cancer) is responsive to said treatment, the treatment is continued.

Also encompassed is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and
wherein differential transcription and/or expression and/or activity level of the gene panel, in the biological sample, relative to the level of corresponding transcription and/or expression and/or activity level of the gene panel determined previously, is predictive of the patient’s response to said treatment.

In a preferred aspect, the gene panel further comprises at least two genes selected among those listed in Table 2, preferably a combination of three or more thereof, preferably a combination of five or more thereof, preferably a combination of ten or more thereof, preferably a combination of twenty or more thereof, preferably a combination of thirty or more thereof, or preferably a combination of all the genes listed in Table 2.

In one aspect, the least one gene selected among the Extra Cellular Matrix (ECM) cluster (Table 2) is selected from the group comprising COL14A1, DOCK1 and ADAMTS2, or a combination thereof.

In one aspect, the patient is predicted as responsive when the level of corresponding transcription and/or expression and/or activity level of the gene panel is downregulated for genes selected from Table 2.

In one aspect, the patient is predicted as not responsive when the level of corresponding transcription and/or expression and/or activity level of the gene panel is upregulated for genes selected from Table 2.

Also encompassed is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among those listed in Table 1, and
wherein differential transcription and/or expression and/or activity level of the gene panel, in the biological sample, relative to the level of corresponding transcription and/or expressionand/or activity level of the gene panel determined previously, is predictive of the patient’s response to said treatment.

Preferably, the gene panel further comprises at least two genes selected among those listed in Table 1, preferably a combination of three or more thereof, preferably a combination of five or more thereof, preferably a combination of ten or more thereof, preferably a combination of twenty or more thereof, preferably a combination of thirty or more thereof, preferably a combination of forty or more thereof, preferably a combination of fifty or more thereof, preferably a combination of sixty or more thereof, preferably a combination of seventy or more thereof, preferably a combination of eighty or more thereof, preferably a combination of ninety or more thereof, or preferably a combination of all the genes listed in Table 1.

In one aspect, the at least one gene selected among those listed in Table 1 is selected from the group comprising MORN4A, BNIPL, CCDC40, and DHRS2, or a combination of two or more thereof, e.g. BNIPL and CCDC40, BNIPL and DHRS2, or DHRS2 and CCDC40. In one aspect, the least one gene selected among those listed in Table 1 consists of the combination of MORN4A, BNIPL, CCDC40, and DHRS2.

In one aspect, the patient is predicted to respond when the level of corresponding transcription and/or expression and/or activity level of the gene panel is downregulated for genes selected from Table 1.

In one aspect, the patient is predicted not to respond when the level of corresponding transcription and/or expression and/or activity level of the gene panel is upregulated for genes selected from Table 1.

Also encompassed is a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the cAMP cluster (Table 3),
wherein differential transcription and/or expression and/or activity level of the gene panel, in the biological sample, relative to the level of corresponding transcription and/or expression and/or activity level of the gene panel determined previously, is predictive of the patient’s response to said treatment.

In a preferred aspect, the gene panel further comprises at least one gene selected among those listed in Table 3, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, or preferably a combination of twelve or more thereof, preferably a combination of thirteen or more thereof, preferably a combination of fourteen or more thereof, or preferably a combination of all the genes listed in Table 3.

In one aspect, said at the least one gene selected among the cAMP cluster (Table 3) is selected from the group comprising PDE10A, CASR, and KCNJ6, or a combination of two or more thereof, e.g. PDE10A and CASR, PDE10A and KCNJ6, or CASR, and KCNJ6. In one aspect, the least one gene selected among the cAMP cluster (Table 3) consists of the combination of PDE10A, CASR, and KCNJ6.

In one aspect, the patient is predicted to respond to the treatment when the level of corresponding transcription and/or expression and/or activity level of the gene panel is downregulated for genes selected from Table 3.

In one aspect, the patient is predicted not to respond to the treatment when the level of corresponding transcription and/or expression and/or activity level of the gene panel is upregulated for genes selected from Table 3.

The present invention further encompasses a computer-implemented method for implementing a method for predicting if a patient having a predetermined disease will respond to a treatment based on immune checkpoint blockade therapy or treatment (ICBT) as described herein, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined previously, whereby differential transcription and/or expression and/or activity level of the gene panel, in the biological sample, relative to the level of corresponding transcription and/or expression and/or activity level of the gene panel determined previously, is predictive of the patient’s response to said treatment.

As used herein, scoring the level of transcription and/or expression and/or activity of a gene panel means transforming the gene expression level of several genes of a panel into a score, with one of the methods described above.

The present invention further encompasses a computer-implemented method for implementing a method for determining if a patient having a predetermined disease is responsive to a treatment based on immune checkpoint blockade therapy or treatment (ICBT) as described herein, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined in a control biological sample, wherein differential transcription and/or expression and/or activity level of the gene panel, in the biological sample of the patient, relative to the level of corresponding transcription and/or expression and/or activity level of the gene panel determined previously, is indicative of whether the patient will respond or not to said treatment.

Within the identified transcripts, the Inventors identified 6 panels. Three panels (table 1, 2, 3) correlate negatively with the prediction of response to ICBT before the start of the therapy. Three other panels (tables 4, 5, 6) correlate mainly positively with a response to ICBT during the therapy.

TABLE 1

92 unique genes from the 105-gene panel identified comparing ICBT responders (Res), or complete responders (ComRes) versus non-responders (NoRes) at baseline. Log2FC: log2 fold change; padj: adjusted p-value

| | | Res vs NoRes | | ComRes vs NoRes | | |
|---|---|---|---|---|---|---|
| Gene ID | Gene symbol | log2FC | padj | log2FC | padj | Ranking |
| ENSG00000171160 | MORN4 | -1.55 | 6.59E-04 | -1.73 | 3.1E-01 | 1 |
| ENSG00000100867 | DHRS2 | -1.32 | 1.23E-03 | -1.75 | 2.6E-01 | 2 |
| ENSG00000163141 | BNIPL | -1.15 | 1.29E-03 | -1.65 | 7.6E-02 | 2 |
| ENSG00000141519 | CCDC40 | -1.25 | 2.91E-03 | -1.38 | 3.9E-01 | 2 |
| ENSG00000163531 | NFASC | -2.23 | 1.86E-05 | -1.91 | 3.9E-01 | 3 |
| ENSG00000122367 | LDB3 | -2.00 | 7.40E-05 | -1.87 | 3.8E-01 | 3 |
| ENSG00000006071 | ABCC8 | -2.53 | 2.35E-04 | -2.48 | 3.4E-01 | 3 |
| ENSG00000228021 | RP11-383C5.3 | -2.44 | 2.35E-04 | -2.33 | 4.1E-01 | 3 |
| ENSG00000115112 | TFCP2L1 | -1.97 | 2.49E-04 | -1.86 | 4.2E-01 | 3 |
| ENSG00000278996 | CH507-513H4.1 | -2.65 | 3.25E-04 | -2.33 | 4.4E-01 | 3 |
| ENSG00000137491 | SLCO2B1 | -2.91 | 3.33E-04 | -2.63 | 4.4E-01 | 3 |
| ENSG00000165655 | ZNF503 | -1.14 | 3.33E-04 | -1.05 | 3.8E-01 | 3 |
| ENSG00000196581 | AJAP1 | -2.97 | 3.33E-04 | -3.16 | 3.8E-01 | 3 |
| ENSG00000274422 | LL22NC03-2H8.5 | -2.27 | 3.33E-04 | -1.91 | 4.0E-01 | 3 |
| ENSG00000132932 | ATP8A2 | -1.94 | 3.53E-04 | -1.72 | 4.4E-01 | 3 |
| ENSG00000115461 | IGFBP5 | -1.78 | 3.65E-04 | -1.94 | 2.6E-01 | 3 |
| ENSG00000163898 | LIPH | -1.22 | 3.89E-04 | -1.51 | 3.2E-01 | 3 |
| ENSG00000250208 | FZD10-AS1 | -2.63 | 4.78E-04 | -3.06 | 3.3E-01 | 3 |
| ENSG00000006042 | TMEM98 | -1.60 | 5.14E-04 | -1.68 | 3.7E-01 | 3 |
| ENSG00000132297 | HHLA1 | -1.74 | 5.14E-04 | -1.64 | 4.4E-01 | 3 |
| ENSG00000265992 | ESRG | -1.85 | 5.14E-04 | -1.92 | 3.8E-01 | 3 |
| ENSG00000285108 | ENSG00000285108 | -1.84 | 5.27E-04 | -1.80 | 4.2E-01 | 3 |
| ENSG00000122966 | CIT | -1.74 | 6.09E-04 | -1.66 | 4.0E-01 | 3 |
| ENSG00000164867 | NOS3 | -1.29 | 6.09E-04 | -1.32 | 3.8E-01 | 3 |
| ENSG00000171914 | TLN2 | -2.05 | 6.09E-04 | -1.42 | 4.4E-01 | 3 |
| ENSG00000105357 | MYH14 | -1.93 | 6.59E-04 | -2.26 | 3.3E-01 | 3 |
| ENSG00000205611 | RP4-610C12.4 | -2.13 | 7.18E-04 | -1.91 | 4.4E-01 | 3 |
| ENSG00000253103 | RP11-946L20.4 | -1.38 | 7.58E-04 | -1.50 | 3.9E-01 | 3 |
| ENSG00000284809 | NA | -2.64 | 9.31E-04 | -2.84 | 4.2E-01 | 3 |
| ENSG00000109832 | DDX25 | -1.98 | 9.40E-04 | -2.09 | 3.8E-01 | 3 |
| ENSG00000234350 | AC007405.4 | -2.05 | 1.00E-03 | -2.08 | 4.0E-01 | 3 |
| ENSG00000173210 | ABLIM3 | -1.49 | 1.00E-03 | -1.75 | 3.4E-01 | 3 |
| ENSG00000285799 | ENSG00000285799 | -1.52 | 1.00E-03 | -1.76 | 3.8E-01 | 3 |
| ENSG00000171680 | PLEKHG5 | -1.87 | 1.08E-03 | -1.81 | 4.4E-01 | 3 |
| ENSG00000197106 | SLC6A17 | -1.78 | 1.15E-03 | -1.76 | 4.4E-01 | 3 |
| ENSG00000253604 | RP11-489E7.1 | -2.53 | 1.15E-03 | -2.60 | 4.4E-01 | 3 |
| ENSG00000144908 | ALDH1L1 | -1.52 | 1.20E-03 | -1.74 | 3.8E-01 | 3 |
| ENSG00000232973 | CYP1B1-AS1 | -1.18 | 1.20E-03 | -1.24 | 4.4E-01 | 3 |
| ENSG00000091622 | PITPNM3 | -1.93 | 1.22E-03 | -1.97 | 4.2E-01 | 3 |
| ENSG00000147724 | FAM135B | -1.84 | 1.24E-03 | -1.57 | 3.4E-01 | 3 |
| ENSG00000149948 | HMGA2 | -1.52 | 1.26E-03 | -1.61 | 3.9E-01 | 3 |
| ENSG00000092621 | PHGDH | -1.27 | 1.41E-03 | -1.29 | 4.4E-01 | 3 |
| ENSG00000275367 | RP11-266K4.14 | -1.68 | 1.41E-03 | -1.73 | 4.3E-01 | 3 |
| ENSG00000234899 | SOX9-AS1 | -1.64 | 1.41E-03 | -1.54 | 4.4E-01 | 3 |
| ENSG00000243742 | RPLP0P2 | -1.26 | 1.41E-03 | -1.49 | 3.8E-01 | 3 |
| ENSG00000242866 | STRC | -1.51 | 1.57E-03 | -1.59 | 4.4E-01 | 3 |
| ENSG00000147862 | NFIB | -1.43 | 1.63E-03 | -1.62 | 3.8E-01 | 3 |
| ENSG00000117266 | CDK18 | -1.38 | 2.15E-03 | -1.36 | 4.4E-01 | 3 |
| ENSG00000141485 | SLC13A5 | -1.59 | 2.21E-03 | -1.75 | 4.2E-01 | 3 |
| ENSG00000144857 | BOC | -1.85 | 2.21E-03 | -2.31 | 3.3E-01 | 3 |
| ENSG00000213279 | RP1-29C18.9 | -1.35 | 2.21E-03 | -1.46 | 4.2E-01 | 3 |
| ENSG00000158220 | ESYT3 | -1.77 | 2.22E-03 | -1.99 | 3.9E-01 | 3 |
| ENSG00000158747 | NBL1 | -1.29 | 2.38E-03 | -1.39 | 4.0E-01 | 3 |
| ENSG00000161714 | PLCD3 | -1.33 | 2.64E-03 | -1.83 | 1.8E-01 | 3 |
| ENSG00000232732 | AC073043.1 | -1.05 | 2.64E-03 | -1.57 | 2.6E-02 | 3 |
| ENSG00000125804 | FAM182A | -1.76 | 2.90E-03 | -1.96 | 4.2E-01 | 3 |
| ENSG00000183914 | DNAH2 | -1.30 | 2.94E-03 | -1.48 | 3.8E-01 | 3 |
| ENSG00000002746 | HECW1 | -2.49 | 3.02E-03 | -3.16 | 3.0E-01 | 3 |
| ENSG00000260596 | DUX4 | -1.01 | 3.02E-03 | -1.17 | 3.8E-01 | 3 |
| ENSG00000062038 | CDH3 | -1.43 | 3.28E-03 | -1.61 | 3.8E-01 | 3 |
| ENSG00000154917 | RAB6B | -1.28 | 3.53E-03 | -1.78 | 1.7E-01 | 3 |
| ENSG00000182175 | RGMA | -1.97 | 3.56E-03 | -2.08 | 3.8E-01 | 3 |
| ENSG00000221813 | OR6B1 | -1.25 | 3.56E-03 | -1.44 | 4.3E-01 | 3 |
| ENSG00000162896 | PIGR | -1.57 | 3.74E-03 | -1.91 | 3.8E-01 | 3 |
| ENSG00000226203 | RP4-760C5.5 | -1.42 | 3.89E-03 | -1.63 | 3.8E-01 | 3 |
| ENSG00000232756 | RP5-118517.1 | -1.67 | 4.03E-03 | -1.70 | 4.4E-01 | 3 |
| ENSG00000152766 | ANKRD22 | -1.32 | 4.04E-03 | -1.71 | 3.6E-01 | 3 |
| ENSG00000187391 | MAGI2 | -1.84 | 4.04E-03 | -1.74 | 4.4E-01 | 3 |
| ENSG00000163435 | ELF3 | -1.33 | 4.06E-03 | -1.55 | 4.3E-01 | 3 |
| ENSG00000085831 | TTC39A | -1.18 | 5.12E-03 | -1.36 | 4.2E-01 | 3 |
| ENSG00000115556 | PLCD4 | -1.09 | 5.17E-03 | -1.33 | 4.2E-01 | 3 |
| ENSG00000196218 | RYR1 | -2.20 | 5.35E-03 | -2.50 | 4.1E-01 | 3 |
| ENSG00000284095 | ENSG00000284095 | -1.00 | 5.35E-03 | -1.11 | 4.4E-01 | 3 |
| ENSG00000099260 | PALMD | -1.20 | 5.42E-03 | -1.32 | 4.4E-01 | 3 |
| ENSG00000279091 | RP11-461F11.2 | -1.40 | 5.42E-03 | -1.59 | 4.4E-01 | 3 |
| ENSG00000121446 | RGSL1 | -1.40 | 5.71E-03 | -1.55 | 4.3E-01 | 3 |
| ENSG00000285591 | ENSG00000285591 | -1.25 | 5.90E-03 | -1.43 | 4.4E-01 | 3 |
| ENSG00000205683 | DPF3 | -1.47 | 6.61E-03 | -1.72 | 4.3E-01 | 3 |
| ENSG00000282024 | ENSG00000282024 | -1.46 | 6.97E-03 | -1.65 | 4.4E-01 | 3 |
| ENSG00000244242 | IFITM10 | -1.26 | 7.09E-03 | -1.83 | 2.6E-01 | 3 |
| ENSG00000165323 | FAT3 | -1.65 | 7.12E-03 | -2.00 | 3.6E-01 | 3 |
| ENSG00000152760 | TCTEX1D1 | -1.45 | 7.17E-03 | -1.61 | 3.8E-01 | 3 |
| ENSG00000163697 | APBB2 | -1.30 | 7.27E-03 | -1.45 | 4.4E-01 | 3 |
| ENSG00000255234 | RP11-727A23.10 | -1.24 | 7.46E-03 | -1.49 | 4.4E-01 | 3 |
| ENSG00000164638 | SLC29A4 | -1.22 | 7.97E-03 | -1.56 | 3.8E-01 | 3 |
| ENSG00000136231 | IGF2BP3 | -1.00 | 8.03E-03 | -1.21 | 4.4E-01 | 3 |
| ENSG00000172403 | SYNPO2 | -1.16 | 8.29E-03 | -1.45 | 4.3E-01 | 3 |
| ENSG00000117152 | RGS4 | -1.28 | 8.39E-03 | -1.49 | 4.2E-01 | 3 |
| ENSG00000102362 | SYTL4 | -1.16 | 8.49E-03 | -1.44 | 4.0E-01 | 3 |
| ENSG00000102098 | SCML2 | -1.15 | 8.83E-03 | -1.25 | 4.4E-01 | 3 |
| ENSG00000139973 | SYT16 | -2.46 | 3.87E-05 | -2.56 | 3.0E-01 | 3 |
| ENSG00000143195 | ILDR2 | -1.95 | 8.06E-05 | -1.88 | 3.8E-01 | 3 |

TABLE 2

Extra Cellular Matrix cluster identified comparing ICBT responders (Res), or complete responders (ComRes) versus non-responders (NoRes) at baseline. Log2FC: log2 fold change; padj: adjusted p-value

| Gene ID | Gene symbol | Res vs NoRes | | ComRes vs NoRes | | |
|---|---|---|---|---|---|---|
| | | log2FC | padj | log2FC | padj | Ranking |
| ENSG00000187955 | COL14A1 | -1.56 | 2.35E-04 | -1.52 | 3.82E-01 | 1 |
| ENSG00000087116 | ADAMTS2 | -2.11 | 1.86E-05 | -1.95 | 3.35E-01 | 2 |
| ENSG00000150760 | DOCK1 | -2.25 | 2.35E-04 | -1.89 | 4.30E-01 | 2 |
| ENSG00000114270 | COL7A1 | -2.12 | 1.86E-04 | -2.48 | 1.39E-01 | 3 |
| ENSG00000142798 | HSPG2 | -1.80 | 5.14E-04 | -1.88 | 3.82E-01 | 3 |
| ENSG00000131711 | MAP1B | -2.29 | 1.86E-04 | -2.41 | 3.32E-01 | 4 |
| ENSG00000112769 | LAMA4 | -1.49 | 7.58E-04 | -1.51 | 4.28E-01 | 4 |
| ENSG00000169894 | MUC3A | -2.70 | 7.58E-04 | -2.45 | 4.43E-01 | 4 |
| ENSG00000130226 | DPP6 | -2.23 | 1.34E-03 | -1.80 | 4.88E-01 | 4 |
| ENSG00000157851 | DPYSL5 | -1.98 | 1.46E-03 | NA | NA | 4 |
| ENSG00000215182 | MUC5AC | -2.31 | 2.40E-03 | -1.69 | 5.10E-01 | 4 |
| ENSG00000116147 | TNR | -1.63 | 2.61E-03 | -1.63 | 4.57E-01 | 4 |
| ENSG00000185518 | SV2B | -1.92 | 2.74E-03 | -1.67 | 5.04E-01 | 4 |
| ENSG00000137809 | ITGA11 | -1.41 | 3.60E-03 | -1.69 | 3.82E-01 | 4 |
| ENSG00000108379 | WNT3 | -1.36 | 3.82E-03 | -1.33 | 4.66E-01 | 4 |
| ENSG00000145113 | MUC4 | -2.47 | 4.26E-03 | -1.94 | 4.88E-01 | 4 |
| ENSG00000122012 | SV2C | -1.60 | 4.40E-03 | -1.79 | 4.04E-01 | 4 |
| ENSG00000148357 | HMCN2 | -1.79 | 4.76E-03 | -1.46 | 4.88E-01 | 4 |
| ENSG00000053747 | LAMA3 | -1.62 | 5.35E-03 | -1.64 | 4.79E-01 | 4 |
| ENSG00000095587 | TLL2 | -1.20 | 5.35E-03 | -1.17 | 4.88E-01 | 4 |
| ENSG00000106278 | PTPRZ1 | -1.40 | 5.63E-03 | -1.43 | 4.79E-01 | 4 |
| ENSG00000142156 | COL6A1 | -1.70 | 5.63E-03 | -1.78 | 4.45E-01 | 4 |
| ENSG00000173269 | MMRN2 | -1.64 | 6.29E-03 | -1.45 | 4.90E-01 | 4 |
| ENSG00000187498 | COL4A1 | -1.44 | 7.25E-03 | -1.54 | 4.64E-01 | 4 |
| ENSG00000184347 | SLIT3 | -1.12 | 7.95E-03 | -1.03 | 5.09E-01 | 4 |
| ENSG00000130635 | COL5A1 | -2.02 | 8.68E-03 | -1.73 | 5.12E-01 | 4 |
| ENSG00000101680 | LAMA1 | -1.12 | 8.86E-03 | -1.11 | 4.88E-01 | 4 |
| ENSG00000134871 | COL4A2 | -1.14 | 8.92E-03 | -0.97 | 5.14E-01 | 4 |
| ENSG00000166147 | FBN1 | -1.31 | 9.71E-03 | -1.32 | 4.88E-01 | 4 |
| ENSG00000113657 | DPYSL3 | -1.53 | 4.18E-02 | -1.27 | 5.52E-01 | 4 |
| ENSG00000087245 | MMP2 | -1.09 | 5.05E-02 | -1.35 | 4.48E-01 | 4 |
| ENSG00000181143 | MUC16 | -1.90 | 1.14E-01 | -0.94 | 7.46E-01 | 4 |
| ENSG00000148848 | ADAM12 | -0.82 | 3.69E-01 | -0.15 | 9.59E-01 | 4 |
| ENSG00000169031 | COL4A3 | -0.28 | 7.20E-01 | -0.44 | 7.82E-01 | 4 |
| ENSG00000078018 | MAP2 | NA | NA | NA | NA | 4 |
| ENSG00000115705 | TPO | NA | NA | NA | NA | 4 |
| ENSG00000135424 | ITGA7 | NA | NA | NA | NA | 4 |
| ENSG00000162733 | DDR2 | NA | NA | -1.45 | 5.12E-01 | 4 |
| ENSG00000197565 | COL4A6 | NA | NA | NA | NA | 4 |

TABLE 3

cAMP cluster identified comparing ICBT responders (Res), or complete responders (ComRes) versus non-responders (NoRes) at baseline. Log2FC: log2 fold change; padj: adjusted p-value

| Gene ID | Gene symbol | Res vs NoRes | | ComRes vs NoRes | | |
|---|---|---|---|---|---|---|
| | | log2FC | padj | log2FC | padj | Ranking |
| ENSG00000112541 | PDE10A | -3.54 | 1.86E-05 | -1.84 | 4.88E-01 | 1 |
| ENSG00000157542 | KCNJ6 | -1.09 | 1.33E-03 | -1.11 | 4.43E-01 | 1 |
| ENSG00000036828 | CASR | -1.37 | 9.99E-04 | -1.64 | 3.56E-01 | 2 |
| ENSG00000173175 | ADCY5 | -2.51 | 1.23E-03 | NA | NA | 3 |
| ENSG00000164742 | ADCY1 | -2.28 | 1.81E-03 | -1.68 | 5.18E-01 | 3 |
| ENSG00000128655 | PDE11A | -1.73 | 2.97E-03 | -2.03 | 3.79E-01 | 3 |
| ENSG00000078295 | ADCY2 | -1.05 | 5.63E-03 | -0.94 | 4.93E-01 | 3 |
| ENSG00000058335 | RASGRF1 | -1.89 | 7.20E-03 | -1.59 | 4.88E-01 | 3 |
| ENSG00000173805 | HAP1 | -1.61 | 7.56E-03 | -0.84 | 6.71E-01 | 3 |
| ENSG00000124493 | GRM4 | -1.72 | 7.97E-03 | -1.10 | 5.69E-01 | 3 |
| ENSG00000111181 | SLC6A12 | -0.94 | 8.38E-03 | -1.19 | 4.18E-01 | 3 |
| ENSG00000182256 | GABRG3 | -2.06 | 1.37E-02 | -0.05 | 9.86E-01 | 3 |
| ENSG00000136928 | GABBR2 | -2.30 | 1.89E-02 | -1.58 | 4.88E-01 | 3 |
| ENSG00000273079 | GRIN2B | -2.43 | 6.42E-02 | -3.59 | 3.79E-01 | 3 |
| ENSG00000183454 | GRIN2A | 0.50 | 7.80E-01 | 0.69 | 8.70E-01 | 3 |

TABLE 4

DNA replication cluster identified comparing ICBT responders (Res), or complete responders (ComRes) before and during therapy. Log2FC: log2 fold change; padj: adjusted p-value

| Gene ID | Gene symbol | Pre vs Post, Res | | Pre vs Post, ComRes | | |
|---|---|---|---|---|---|---|
| | | log2FC | padj | log2FC | padj | Ranking |
| ENSG00000142731 | PLK4 | 1.04 | 9.06E-02 | 0.98 | 8.14E-01 | 1 |
| ENSG00000138778 | CENPE | 1.13 | 2.98E-01 | 0.05 | 9.99E-01 | 1 |
| ENSG00000184661 | CDCA2 | 1.44 | 3.13E-02 | 1.67 | 3.35E-01 | 2 |
| ENSG00000129173 | E2F8 | 1.67 | 4.61E-02 | 2.21 | 4.36E-01 | 2 |
| ENSG00000131747 | TOP2A | 0.97 | 2.11E-02 | 1.50 | 1.60E-03 | 2 |
| ENSG00000126787 | DLGAP5 | 0.99 | 1.95E-02 | 1.30 | 1.41E-01 | 2 |
| ENSG00000163535 | SGOL2 | 1.11 | 6.93E-02 | 1.65 | 9.61E-02 | 2 |
| ENSG00000074181 | NOTCH3 | 1.08 | 1.16E-01 | 1.20 | 8.74E-01 | 2 |
| ENSG00000157456 | CCNB2 | 0.80 | 1.19E-01 | 1.43 | 2.26E-01 | 2 |
| ENSG00000066279 | ASPM | 0.65 | 8.27E-02 | 1.05 | 1.33E-01 | 2 |
| ENSG00000072501 | SMC1A | 0.69 | 2.44E-02 | 0.71 | 5.21E-01 | 2 |
| ENSG00000065328 | MCM10 | 1.16 | 8.01E-02 | 0.55 | 9.99E-01 | 3 |

TABLE 5

Interferon cluster identified comparing ICBT responders (Res), or complete responders (ComRes) before and during therapy. Log2FC: log2 fold change; padj: adjusted p-value

| Gene ID | Gene symbol | Pre vs Post, Res | | Pre vs Post, ComRes | | |
|---|---|---|---|---|---|---|
| | | log2FC | padj | log2FC | padj | Ranking |
| ENSG00000188389 | PDCD1 | 0.99 | 2.49E-02 | 1.36 | 5.40E-01 | 1 |
| ENSG00000105205 | CLC | 0.96 | 1.35E-01 | 1.48 | 7.10E-01 | 1 |
| ENSG00000123609 | NMI | 0.23 | 4.36E-01 | 0.66 | 4.16E-02 | 1 |
| ENSG00000150337 | FCGR1A | 0.72 | 8.83E-02 | 1.59 | 8.06E-09 | 2 |
| ENSG00000198019 | FCGR1B | 0.56 | 1.91E-01 | 1.37 | 1.01E-05 | 2 |
| ENSG00000121380 | BCL2L14 | 1.03 | 7.54E-02 | 1.24 | 7.12E-01 | 2 |
| ENSG00000142089 | IFITM3 | 0.31 | 6.17E-01 | 1.30 | 5.14E-03 | 2 |
| ENSG00000115415 | STAT1 | 0.35 | 3.47E-01 | 0.94 | 5.14E-03 | 2 |
| ENSG00000162645 | GBP2 | 0.34 | 3.10E-01 | 0.87 | 1.60E-03 | 2 |
| ENSG00000106804 | C5 | 0.81 | 8.97E-02 | 1.15 | 3.00E-01 | 2 |
| ENSG00000152778 | IFIT5 | 0.33 | 4.22E-01 | 1.00 | 1.86E-02 | 2 |
| ENSG00000068079 | IFI35 | 0.19 | 7.16E-01 | 0.94 | 4.80E-02 | 2 |
| ENSG00000132274 | TRIM22 | 0.32 | 3.77E-01 | 0.90 | 3.28E-02 | 2 |
| ENSG00000136634 | IL10 | 0.87 | 1.02E-01 | 1.26 | 6.56E-01 | 2 |
| ENSG00000131203 | IDO1 | 0.95 | 8.27E-02 | 1.24 | 9.80E-01 | 2 |

TABLE 6

45 unique genes from the 51-gene panel identified comparing ICBT responders (Res), or complete responders (ComRes) before and during therapy. Log2FC: log2 fold change; padj: adjusted p-value

| Gene ID | Gene symbol | Pre-Post Res | | Pre-Post ComResp | | Ranking |
|---|---|---|---|---|---|---|
| | | log2FC | padj | log2FC | padj | |
| ENSG00000196664 | TLR7 | -0.89 | 3.01E-03 | -0.64 | 4.87E-01 | 1 |
| ENSG00000143156 | NME7 | 0.56 | 1.25E-02 | 0.61 | 3.30E-01 | 1 |
| ENSG00000108175 | ZMIZ1 | -0.66 | 2.44E-02 | -0.38 | 7.55E-01 | 1 |
| ENSG00000066468 | FGFR2 | 0.92 | 3.01E-03 | 0.75 | 4.97E-01 | 2 |
| ENSG00000137491 | SLCO2B1 | 1.91 | 3.01E-03 | 1.04 | 8.06E-01 | 2 |
| ENSG00000124785 | NRN1 | 1.17 | 1.18E-02 | 0.81 | 7.10E-01 | 2 |
| ENSG00000156113 | KCNMA1 | 0.99 | 1.40E-02 | 0.47 | 9.99E-01 | 2 |
| ENSG00000106144 | CASP2 | -0.43 | 1.95E-02 | -0.34 | 8.05E-01 | 2 |
| ENSG00000186094 | AGBL4 | 1.20 | 1.95E-02 | 0.79 | 9.99E-01 | 2 |
| ENSG00000104774 | MAN2B1 | -0.37 | 2.44E-02 | -0.28 | 9.25E-01 | 2 |
| ENSG00000147036 | LANCL3 | 0.63 | 2.44E-02 | 0.62 | 8.52E-01 | 2 |
| ENSG00000176641 | RNF152 | 0.86 | 2.44E-02 | 0.68 | 9.14E-01 | 2 |
| ENSG00000163792 | TCF23 | 0.91 | 2.44E-02 | 0.60 | 9.99E-01 | 2 |
| ENSG00000282556 | NA | 1.21 | 2.44E-02 | 1.14 | 7.51E-01 | 2 |
| ENSG00000241484 | ARHGAP8 | 1.28 | 2.44E-02 | 0.74 | 9.99E-01 | 2 |
| ENSG00000196581 | AJAP1 | 1.50 | 2.44E-02 | 1.13 | 7.85E-01 | 2 |
| ENSG00000182580 | EPHB3 | 1.74 | 2.44E-02 | 0.62 | 9.99E-01 | 2 |
| ENSG00000273079 | GRIN2B | 3.19 | 2.44E-02 | 1.55 | 9.99E-01 | 2 |
| ENSG00000276203 | ANKRD20A3 | 1.10 | 2.49E-02 | 0.96 | 6.27E-01 | 2 |
| ENSG00000171435 | KSR2 | 1.62 | 2.49E-02 | 1.63 | 1.52E-01 | 2 |
| ENSG00000113645 | WWC1 | 1.47 | 2.54E-02 | -0.06 | 9.99E-01 | 2 |
| ENSG00000085276 | MECOM | 1.27 | 2.81E-02 | 0.49 | 9.99E-01 | 2 |
| ENSG00000196943 | NOP9 | -0.38 | 2.82E-02 | -0.34 | 8.22E-01 | 2 |
| ENSG00000139767 | SRRM4 | 1.15 | 2.85E-02 | 0.42 | 9.99E-01 | 2 |
| ENSG00000130649 | CYP2E1 | 1.46 | 3.13E-02 | 0.65 | 9.99E-01 | 2 |
| ENSG00000227036 | LINC00511 | 0.79 | 3.60E-02 | 0.47 | 7.85E-01 | 2 |
| ENSG00000116147 | TNR | 0.90 | 3.60E-02 | 0.32 | 9.99E-01 | 2 |
| ENSG00000157851 | DPYSL5 | 1.08 | 3.60E-02 | 0.97 | 9.99E-01 | 2 |
| ENSG00000112541 | PDE10A | 1.58 | 3.60E-02 | 0.55 | 9.99E-01 | 2 |
| ENSG00000157933 | SKI | -0.80 | 3.74E-02 | -0.39 | 9.99E-01 | 2 |
| ENSG00000127022 | CANX | -0.22 | 3.74E-02 | -0.15 | 9.99E-01 | 2 |
| ENSG00000255339 | RP11-411B6.6 | -0.81 | 3.84E-02 | -0.92 | 6.26E-01 | 2 |
| ENSG00000215458 | AP001053.11 RP11- | -0.46 | 3.84E-02 | -0.41 | 7.81E-01 | 2 |
| ENSG00000258952 | 1042B17.5 | 1.16 | 3.84E-02 | 0.43 | 9.99E-01 | 2 |
| ENSG00000122367 | LDB3 | 1.18 | 3.84E-02 | 0.73 | 9.99E-01 | 2 |
| ENSG00000123095 | BHLHE41 | 1.14 | 3.95E-02 | 1.23 | 7.51E-01 | 2 |
| ENSG00000180902 | D2HGDH NPHP3- | -0.57 | 3.98E-02 | -0.69 | 5.84E-01 | 2 |
| ENSG00000274810 | ACAD11 | -1.32 | 4.09E-02 | -0.76 | 9.99E-01 | 2 |
| ENSG00000150676 | CCDC83 | 1.20 | 4.13E-02 | NA | NA | 2 |
| ENSG00000113657 | DPYSL3 | 1.62 | 4.13E-02 | 0.53 | 9.99E-01 | 2 |
| ENSG00000162591 | MEGF6 RP11- | -0.51 | 4.57E-02 | -0.51 | 6.26E-01 | 2 |
| ENSG00000277128 | 146E13.4 | 0.49 | 4.57E-02 | 0.32 | 9.94E-01 | 2 |
| ENSG00000158220 | ESYT3 | 1.19 | 4.57E-02 | 0.45 | 9.99E-01 | 2 |
| ENSG00000232775 | BMS1P18 | 1.58 | 4.57E-02 | 2.06 | 6.07E-01 | 2 |
| ENSG00000119866 | BCL11A | -0.40 | 4.61E-02 | -0.44 | 6.85E-01 | 2 |

Also encompassed in the present invention is the use of a gene panel comprising at least one gene selected among the Extra Cellular Matrix (ECM) cluster (Table 2) and, at least one gene selected among those listed in Table 1, wherein the at least one ECM gene cluster comprises COL14A1 and the at least one gene of Table 1 comprises MORN4A, for predicting if a patient having a predetermined disease will respond to a treatment based on immune checkpoint blockade therapy or treatment (ICBT).

In one aspect, the at least one gene selected among those listed in Table 1 is further selected from the group comprising BNIPL, CCDC40, and DHRS2, or a combination of two or more thereof, e.g. BNIPL and CCDC40, BNIPL and DHRS2, or DHRS2 and CCDC40. In one aspect, the least one gene selected among those listed in Table 1 consists of the combination of MORN4A, BNIPL, CCDC40, and DHRS2.

Preferably, the gene panel further comprises at least two genes selected among those listed in Table 1, preferably a combination of three or more thereof, preferably a combination of five or more thereof, preferably a combination of ten or more thereof, preferably a combination of twenty or more thereof, preferably a combination of thirty or more thereof, preferably a combination of forty or more thereof, preferably a combination of fifty or more thereof, preferably a combination of sixty or more thereof, preferably a combination of seventy or more thereof, preferably a combination of eighty or more thereof, or more preferably a combination of ninety-two thereof.

In one aspect, the least one gene selected among the Extra Cellular Matrix (ECM) cluster (Table 2) is further selected from the group comprising DOCK1 and ADAMTS2, or a combination thereof.

Preferably, the gene panel further comprises at least two genes selected among those listed in Table 2, preferably a combination of three or more thereof, preferably a combination of five or more thereof, preferably a combination of ten or more thereof, preferably a combination of twenty or more thereof, preferably a combination of thirty or more thereof, or more preferably a combination of all the genes listed in Table 2.

In a further aspect, the gene panel further comprises at least one gene selected among the cAMP cluster (Table 3). Preferably, said at the least one gene selected among the cAMP cluster (Table 3) is selected from the group comprising PDE10A, CASR, and KCNJ6, or a combination of two or more thereof, e.g. PDE10A and CASR, PDE10A and KCNJ6, or CASR, and KCNJ6. In one aspect, the least one gene selected among the cAMP cluster (Table 3) consists of the combination of PDE10A, CASR, and KCNJ6.

Preferably, the gene panel further comprises at least one gene selected among those listed in Table 3, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, or preferably a combination of twelve or more thereof, preferably a combination of thirteen or more thereof, preferably a combination of fourteen or more thereof, or preferably a combination of all the genes listed in Table 3.

Also encompassed in the present invention is the use of a gene panel comprising at least one gene selected among the DNA replication cluster (Table 4) and, at least one gene selected among the gene interferon cluster (Table 5), wherein the at least one DNA replication gene cluster comprises PLK4 and the at least one interferon cluster gene comprises PDCD1, for determining if a patient having a predetermined disease is responsive to a treatment based on immune checkpoint blockade therapy or treatment (ICBT).

In one aspect, the least one gene selected among the DNA replication cluster (Table 4) is further selected from the group further comprising CENPE, CDCA2, E2F8, TOP2A, DLGAP5, SGOL2, NOTCH3, CCNB2, ASPM, SMC1A and MCM10, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, or preferably a combination of all the genes listed in Table 4.

In one aspect, the least one gene selected among the gene interferon cluster is selected from the group further comprising CLC, NMI, FCGR1A, FCGR1B, BCL2L14, IFITM3, STAT1, GBP2, C5, IFIT5, IFI35, TRIM22, IL10, and IDO1, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of twelve or more thereof, preferably a combination of thirteen or more thereof, or preferably a combination of all the genes listed in Table 5.

In one aspect, the gene panel further comprises at least one gene selected among those listed in Table 6, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of fifteen or more thereof, preferably a combination of twenty or more thereof, preferably a combination of twenty five or more thereof, preferably a combination of thirty or more thereof, preferably a combination of thirty five or more thereof, preferably a combination of forty or more thereof, or preferably a combination of all the genes listed in Table 6.

Further encompassed is the use of a gene panel for predicting if a patient having a predetermined disease will respond to a treatment based on immune checkpoint blockade therapy or treatment (ICBT), comprising:
at least one gene selected among the Extra Cellular Matrix (ECM) cluster (Table 2) and/or
at least one gene selected among those listed in Table 1, and/or
at least one gene selected among the cAMP cluster (Table 3), and/or
at least one gene selected among the DNA replication cluster of Table 4.

Further encompassed is the use of a gene panel for determining if a patient having a predetermined disease is responsive to a treatment based on immune checkpoint blockade therapy or treatment (ICBT), comprising:
at least one gene selected among the DNA replication cluster of Table 4, and/or
at least one gene selected among the Interferon cluster genes (Table 5), and/or
at least one gene selected among those listed in Table 6.

Also encompassed in the present invention is a kit for performing a method of the invention, said kit comprising
a) means and/or reagents for determining the level of transcription and/or expression and/or activity of said gene panel in a biological sample from said patient, and
b) instructions for use.

Also encompassed in the present invention are methods of treatment.

In one aspect, the method of treatment of a cancer or an autoimmune disease, comprises
i) detecting in a biological sample obtained from said patient the level of transcription and/or expression and/or activity of a gene panel of any one of Tables 1, 2, and/or 3,
ii) and treating the patient based upon whether a differential transcription and/or expression and/or activity level of said gene panel, in the biological sample, relative to the level of corresponding transcription and/or expression and/or activity level of the gene panel determined previously, is predictive of the patient’s response to said treatment.

In one aspect, the method of treatment of a cancer or an autoimmune disease, comprises
i) detecting in a biological sample obtained from said patient the level of transcription and/or expression and/or activity of a gene panel of any one of Tables 4, 5, and/or 6,
ii) and treating the patient based upon whether a differential transcription and/or expression and/or activity level of said gene panel, in the biological sample, relative to the level of corresponding transcription and/or expression and/or activity level of the gene panel determined previously, is predicting that the patient is responsive to said treatment.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### Materials and Methods

### Patients

A retrospective study included 32 patients with mUC who were treated with anti-PD-1 in the Radboud University Medical Center between 2017 and 2019. Patients were treated with nivolumab 3 mg/kg every 2 weeks or pembrolizumab 200 mg every 3 weeks. During treatment, patients were evaluated according to RECIST1.1. Patients were considered to have clinical benefit if they had a radiological and clinical progression-free survival (PFS) of at least 6 months.

All patients provided informed consent for the use of biomaterials as approved by the medical ethics committee of the Radboud University Medical Center (project number NL60249.091.16). This study was performed in accordance with relevant guidelines and regulations.

### Blood Collection and Processing

Blood was drawn prior to the first 2 or 3 cycles of anti-PD-1 therapy (i.e. at 0, 2 or 4 weeks for nivolumab and at 0, 3 or 6 weeks for pembrolizumab). At these timepoints, a complete blood cell count was performed as part of routine clinical care. In addition, blood was collected in one PAXgene Blood RNA tube (BD Biosciences, San Jose, CA, USA). PAXgene tubes were stored at - 80° C. until RNA purification. A baseline sample and the earliest on-treatment sample available was used for subsequent analyses.

### Whole Blood-RNA Sequencing

Total RNA was extracted from whole blood using the PAXgene blood miRNA kit (Qiagen, Venlo, Netherlands). RNA quantity was determined using Qubit (Thermo Fisher Scientific, Waltham, MA, USA). RNA quality was assessed on a Bioanalyzer (Agilent Technologies, Santa Clara, CA, USA). Samples with a RIN below 6 were excluded from analysis. Per sample, at least 500 ng of total RNA was used for library preparation.

RNA samples were treated for globin and ribosomal RNA depletion with the Illumina Globin-Zero Gold kit (Illumina, San Diego, CA, USA). Library preparation was performed with the Illumina TruSeq RNA Library Prep Kit v2. Sequencing was performed on Illumina NovaSeq 6000 (non-stranded, paired-end 2×150 bp) with an estimated average output of 20-30 million reads/sample.

Adapter-trimmed paired-end reads were used as input for gene expression analysis on the LITOSeek platform (Novigenix SA, Epalinges, Switzerland). Reads were aligned to the human reference hg38 with HISAT2 (2.1.0), and the Salmon algorithm (0.13.1) was used to quantify transcript expression. A preliminary quality check was performed using the MultiQC tool (version 1.8).

The quantified transcript expression data was used to identify biomarkers to predict clinical benefit before therapy start as well as early markers of response.

Differential expression analyses (DEA) were performed using DESeq2. Log fold changes and adjusted p-values were determined for all genes using a Wald test with Benjamini-Hochberg correction. Functional enrichment analysis was performed with EnrichR, using the Reactome 2016 database. Network analyses were performed using STRING.

### Bioinformatics and Statistical Analyses

A ranking system for selecting a target panel of features was implemented. The genes’ rank is calculated with a mathematical weighted sum of results from several performed univariate and multivariate statistical methods. The results are for example the fold change, p-adjusted and genes and their coefficients importance in the statistical models.

Sparse partial least squares (SPLS) regression and classification (Chun and Keles (2010) with nearest shrunken centroid method (T. Hastie, R. Tibshirani, Balasubramanian Narasimhan, Gil Chu (2002) was applied on different gene panels for the classification method to generate and test models for predicting clinical benefit to anti-PD-1.

The data was splitting in 70% of training and 30% of test set. The training and parameter optimization was run on the training set only with 10 repeated nested 3-fold cross validation (CV) method. Ten randomized test data splits have been used for performance evaluation of the predictive models. Specificity and sensitivity at different probability score cut-offs were calculated and Receiver Operating Characteristics (ROC) curves generated. Ten randomized data splits have been used for performance evaluation of the predictive models. Specificity and sensitivity at different probability score cut-offs were calculated and Receiver Operating Characteristics (ROC) curves generated.

To calculate genes importance we have used varImp R Package from the Caret (https://cran.r-project.org/web/packages/varImp/varImp.pdf).

Kaplan-Meier curves were generated to display differences in progression free survival (PFS) between patients. A probability score of 0.5 was used to dichotomized patients in responder and non-responder group.

### Results

### Patient Cohort

In total, 32 patients with mUC were included. Most patients were treated with pembrolizumab (78.1%) and received anti-PD-1 as second-line treatment (65.6%). Patient characteristics are summarized in table 7.

Nineteen patients experienced clinical benefit (59.4%). Five of them had a complete response, 12 a partial response, and one had stable disease according to RECIST1.1. Additionally, one patient was non-evaluable according to RECIST1.1 criteria but showed a decrease in FDG uptake on PET imaging. Median PFS in the group with clinical benefit was 25 months (range: 10 - >42). Median OS could not be determined because only two patients had died at last follow up (median follow up: 33 months). By contrast, thirteen patients (40.6%) did not experience clinical benefit. None of these patients had an initial response. In these patients, median PFS and OS were 2 (range: 1-3) and 6 months (range: 1 - 30), respectively.

In most patients, the on-treatment sample was collected after 1 cycle of anti-PD-1 (75%). In total, high-quality RNA-sequencing data of baseline and on-treatment samples was available for 26 of 32 patients (14 with clinical benefit, 12 without clinical benefit). In five patients, either the baseline or on-treatment sample did not pass the quality check. In one patient, no PAXgene tube was available.

TABLE 7

Patient characteristics.

| | |
|---|---|
| Total - n (%) | 32 (100%) |
| Male - n (%) | 25 (78.1) |
| Age - median (range) | 68 (38 - 80) |
| Treatment - n (%) | |
| Nivolumab | 7 (21.9) |
| Pembrolizumab | 25 (78.1) |
| Previous platinum-based chemotherapy - n (%) | 21 (65.6) |
| Clinical outcome - n (%)* | |
| PFS < 6 months | 13 (40.6) |
| PFS ≥ 6 months | 19 (59.4) |
| Complete response | 5 (15.6) |
| Partial response | 12 (37.5) |
| Stable disease | 1(3.1) |
| Not evaluable | 1(3.1) |

* according to RECIST1.1

### Whole Blood Transcriptome Changes in Patients With and Without Clinical Benefit at Baseline

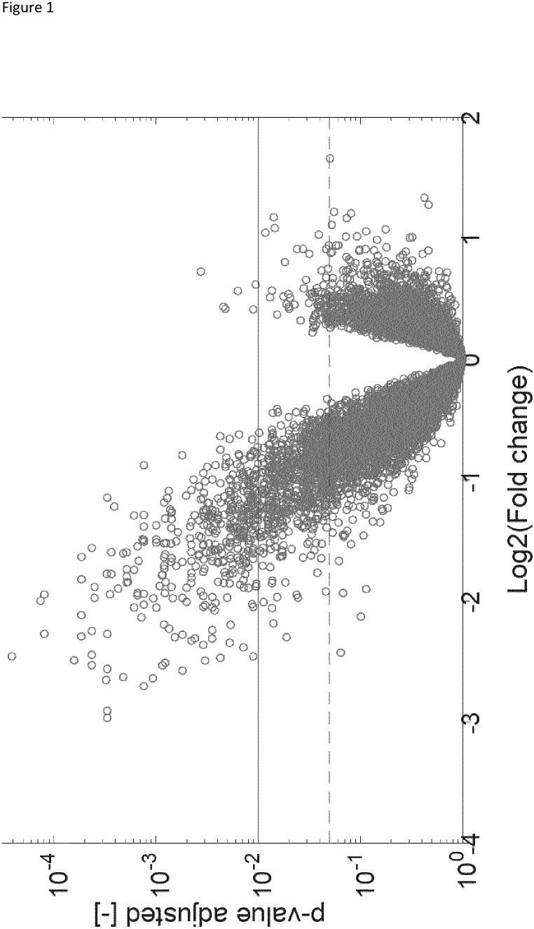

First, a Differential Expression Analysis (DEA) was performed between 18 CB+ and 13 CB-at baseline. We identified 351 differentially expressed genes (DEGs) with an adjusted p-value (padj) < 0.01 and a fold change (FC) >2 (FIG. 1). The great majority of DEGs (n=293) were not expressed in blood from healthy donors. When the DEA was restricted to patients with complete response, 280 DEGs were identified, with 105 DEGs in common between the two analyses. A biological functional analysis of the DEGs identified by the two DEA analyses highlighted a downregulated 39-gene cluster related to the extracellular matrix (ECM) (Table 2) and a downregulated 15-gene cluster related to the cAMP pathway (Table 3). Thirteen genes from the ECM and cAMP cluster are part of the 105-gene panel, which thus include 92 unique genes (table 1).

### Biomarkers Performance for Predicting Response to Therapy

To test the ability of the 105-gene panel as well as the ECM and the cAMP panel to predict the response to anti-PD1 therapy at baseline, we trained predictive models with the 3 different gene panels as input. The performances of the 3 different predictive models are listed in table 8.

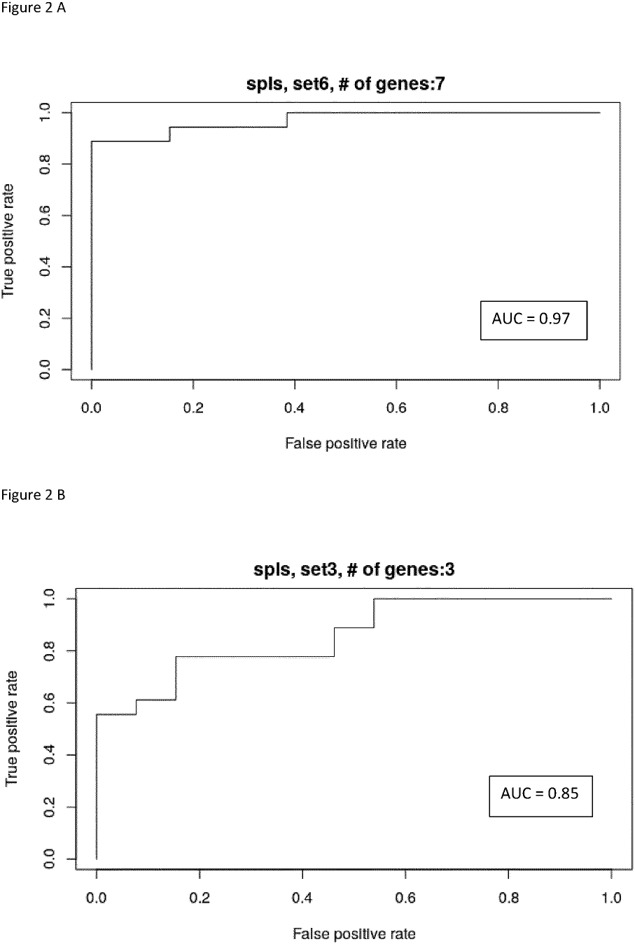

All panels demonstrated high predictive value, with the 105-gene one showing the highest performances: it predicted clinical benefit from anti-PD-1 therapy with an area under the curve (AUC) of 0.95, 89% sensitivity and 68% specificity (table 9, FIGS. 2).

To know which genes contributed the most to the predictive power of the model within each gene list, we computed the gene importance within each trained model, defined as the selection frequency, and we used this parameter to rank the genes in each list.

To test whether the gene panels could be further reduced without compromising the predictive power, we identified 7 gene subsets, defined as the top 3 or 4 genes in each ranked list and their combinations (table 9), and we trained again predictive models for response to anti-PD1 therapy. All the subsets showed excellent predictive value (table 8). In particular subset 6, which is the combination of the top ranked genes of the 105 and the ECM gene panel, showed an AUC of 0.97, 94% sensitivity and 87% specificity. This indicate that this gene subset is an indispensable and key element for predicting response to therapy.

TABLE 8

Gene lists performances

| Gene list | Accuracy | AUC | Balanced Accuracy | Sensitivity | Specificity |
|---|---|---|---|---|---|
| 105 gene-list | 0.81 | 0.95 | 0.79 | 0.89 | 0.68 |
| 92 gene list | 0.78 | 0.94 | 0.75 | 0.89 | 0.62 |
| cAMP 15-gene list | 0.81 | 0.90 | 0.79 | 0.89 | 0.68 |
| ECM 39-gene list | 0.71 | 0.88 | 0.69 | 0.78 | 0.60 |
| ECM + cAMP | 0.71 | 0.89 | 0.68 | 0.83 | 0.53 |
| subset 1 | 0.87 | 0.97 | 0.86 | 0.94 | 0.78 |
| subset 2 | 0.88 | 0.93 | 0.88 | 0.89 | 0.87 |
| subset 3 | 0.75 | 0.85 | 0.74 | 0.78 | 0.70 |
| subset 4 | 0.93 | 0.94 | 0.93 | 0.94 | 0.92 |
| subset 5 | 0.88 | 0.92 | 0.88 | 0.89 | 0.87 |
| subset 6 | 0.91 | 0.97 | 0.91 | 0.94 | 0.87 |
| subset 7 | 0.93 | 0.96 | 0.93 | 0.94 | 0.92 |

TABLE 9

Gene subsets derived from the 105, ECM, cAMP gene panels

| Subset | 1 | 2 | 3 | 4 | 5 | 6 | 7 |
|---|---|---|---|---|---|---|---|
| Gene panel | | | | | | | |
| ECM | COL14A1 | COL14A1 | | | COL14A1 | COL14A1 | |
| ECM | DOCK1 | DOCK1 | | | DOCK1 | DOCK1 | |
| ECM | ADAMTS2 | ADAMTS2 | | | ADAMTS2 | ADAMTS2 | |
| cAMP | KCNJ6 | | KCNJ6 | | KCNJ6 | | KCNJ6 |
| cAMP | CASR | | CASR | | CASR | | CASR |
| cAMP | PDE10A | | PDE10A | | PDE10A | | PDE10A |
| 105 genes | MORN4 | | | MORN4 | | MORN4 | MORN4 |
| 105 genes | BNIPL | | | BNIPL | | BNIPL | BNIPL |
| 105 genes | CCDC40 | | | CCDC40 | | CCDC40 | CCDC40 |
| 105 genes | DHRS2 | | | DHRS2 | | DHRS2 | DHRS2 |

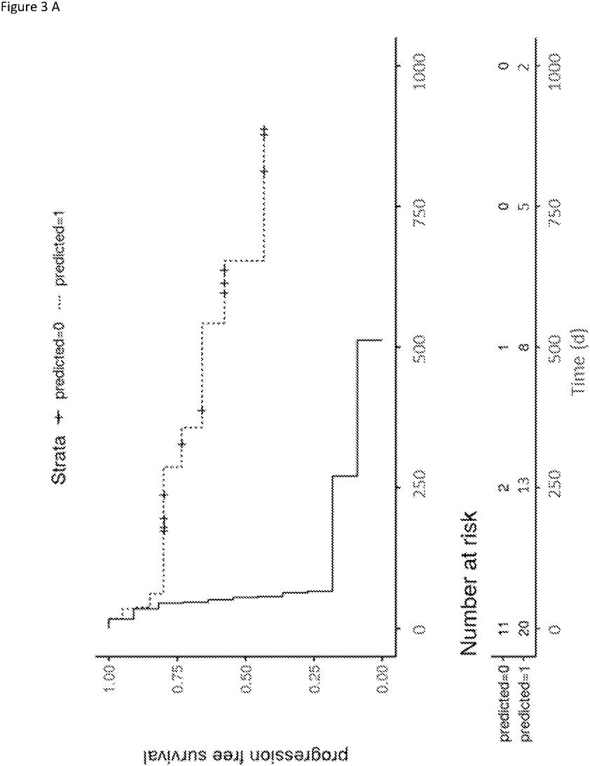

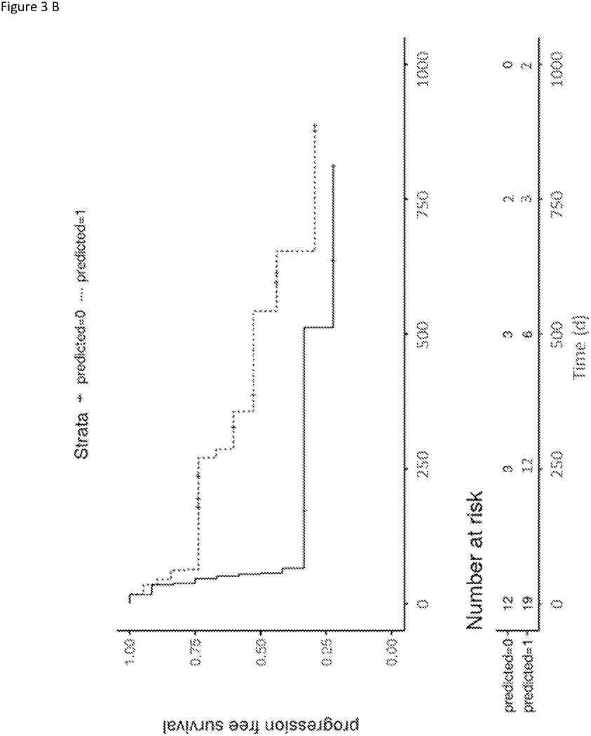

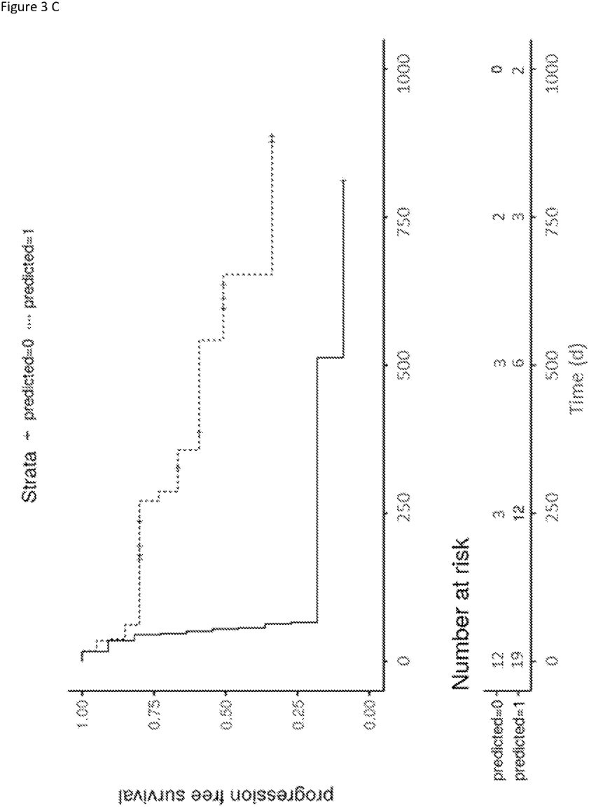

Finally, the patient cohort was stratified according to the classification output of the 105-gene, ECM and cAMP based models and progression-free survival (PFS) curves were generated (FIGS. 3). The group classified as responders showed a clear benefit in the progression-free survival compared to the non-responder group at six months for all 3 gene panels.

### Whole Blood Transcriptome Changes in Patients Before and During Treatment

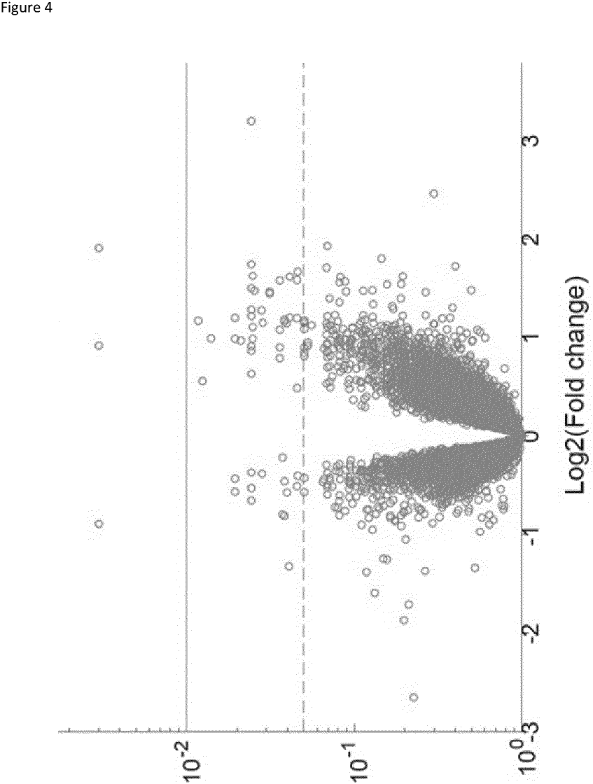

To discover biomarkers for early changes of the response to anti-PD1, a DEA between baseline and on-treatment samples was performed in 14 patients with clinical benefit to anti-PD-1. Fifty-one differentially expressed genes (DEGs) were identified, of which 37 were upregulated and 14 downregulated (FIG. 4). The average fold change of these DEGs was 2.0. For biological interpretation of the identified DEGs, we first generated a protein-protein interaction network using STRING to explore interactions between the identified protein-coding DEGs. Among the 51 DEGs were 43 protein-coding genes. STRING network analysis revealed a cluster of 5 interconnected DEGs which were all involved in DNA replication or cell cycle regulation. Four of these genes were upregulated, i.e., DLGAP5, TOP2A, CDCA2, and E2F8. Changes in the expression of these genes were highly correlated. SMC1A, on the other hand, which is known for its role in chromosome cohesion during the cell cycle, was downregulated and changes in SMC1A poorly correlated with changes in the other DNA replication/cell cycle gene in our cohort. Pathway enrichment analysis did not identify any significantly enriched pathways (padj ≤0.05). As no enriched pathways were identified, we looked further into the function of individual DEGs. Interestingly, we observed that PDCD1, the gene that encodes for PD-1, was upregulated in patients with clinical benefit. Except for PDCD1, the identified DEGs did not have an established role in immunology.

Subsequently, we performed a similar analysis in the 12 patients without clinical benefit. In contrast to the patients with clinical benefit, no DEGs were identified in these patients. Particularly, no net increase or decrease was observed in any of the DNA replication/cell cycle genes that were differentially expressed in the patients with clinical benefit, nor in PDCD1 expression.

In our cohort, 5 patients achieved a complete response. We wondered whether changes in RNA expression might be even more pronounced in these patients. Therefore, we performed a DEA between baseline and on-treatment samples of the 5 complete responders. Thirty-eight DEGs were identified, most of which were upregulated (30/38). Protein-protein interaction analysis revealed many interactions, especially between DEGs related to interferon signaling (false discovery rate: 3.10 × 10-9). In line with this, pathway enrichment analysis revealed that the DEGs were enriched for interferon signaling (overlap 7/196 genes, padj: 7.7 ×10-6) and cytokine signaling genes (overlap 7/620 genes, padj: 0.0037; table 2). All seven interferon/cytokine signaling genes were upregulated (STAT1, IFITM3, TRIM22, GBP2, IFI35, FCGR1B and FCGR1A). A 15-gene interferon (IFN) cluster was compiled by adding to these 7 DEGs all the IFN-related DEGs identified in the DEA with all the responders (table 5).

Similarly, a 12-gene DNA replication cluster was compiled by adding to the 6 DEGs identified in the DEA with all the responders, 6 DNA replication DEGs identified in the DEA with only complete responders (table 4).

Based on the mechanism of action of anti-PD-1 and previously published data describing T-cell reinvigoration in responders to ICI, we hypothesized that the upregulation of DNA replication genes/cell cycle genes in patients with clinical benefit may be partly due to proliferation of peripheral T cells. To evaluate the cell specificity of the identified DNA replication/cell cycle genes, we used a publicly available dataset consisting of RNA-sequencing data of flow cytometry-sorted PBMCs (GSE107011). We observed enhanced expression of DLGAP5, TOP2A, CDCA2, and E2F8 in T cells compared to unsorted PBMCs. Expression was particularly high in CD8⁺ effector memory cells, T-helper 1 cells, follicular helper T cells, and regulatory T cells. SMC1A, on the other hand, was highly expressed in nearly all immune cells subsets, showing no specificity for any particular immune cell subset.

### Biomarkers Performance for Determining Response to Therapy

To test the ability of the 51-gene, DNA replication and the IFN gene panel to predict the response to anti-PD1 therapy at baseline, we trained predictive models using the 3 different gene panels as input. The 51-gene panel had 6 gene in common with the DNA replication and the IFN cluster, and therefore it was reduced to 45 unique genes (table 6). The performances of the 3 different predictive models are listed in table 10.

All panels demonstrated good predictive value, with the DNA replication panel showing the highest performance: it determined an early response to anti-PD-1 therapy with an area under the curve (AUC) of 0.58, and a balanced accuracy of 58% (table 10).

To know which genes contributed the most to the predictive power of the model within each gene list, we computed the gene importance within each trained model, and we used this parameter to rank the genes in each list.

TABLE 10

Gene lists performances

| | Accuracy | AUC | Balanced Accuracy | Sensitivity | Specificity |
|---|---|---|---|---|---|
| DNArep 12 genes | 0.57 | 0.58 | 0.58 | 0.58 | 0.58 |
| 45 genes + DNArep +IFN | 0.49 | 0.63 | 0.52 | 0.37 | 0.67 |
| 45 genes | 0.42 | 0.51 | 0.45 | 0.31 | 0.58 |
| IFN 15 genes | 0.42 | 0.43 | 0.39 | 0.53 | 0.25 |

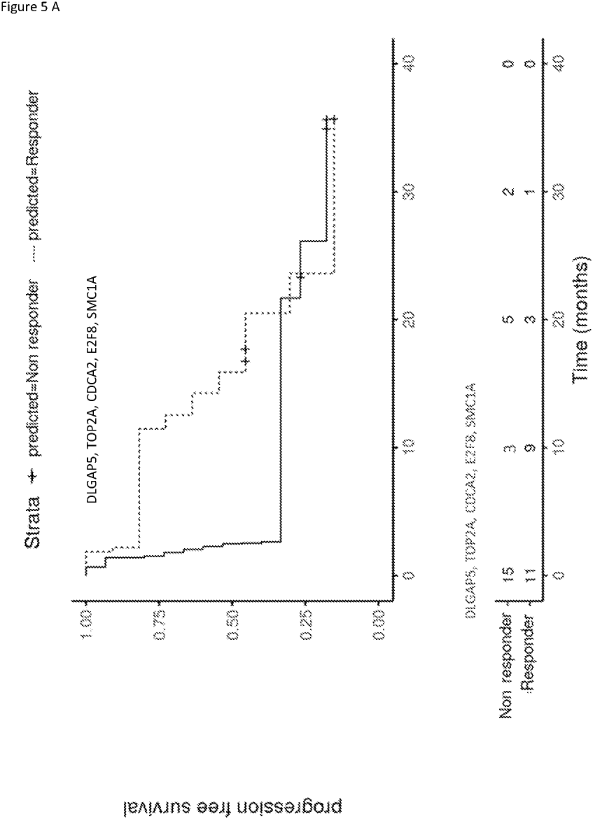

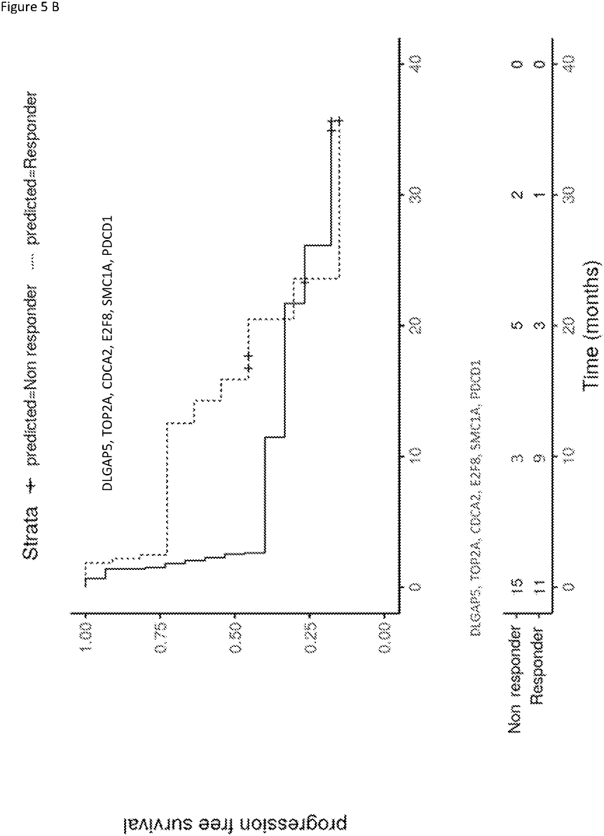

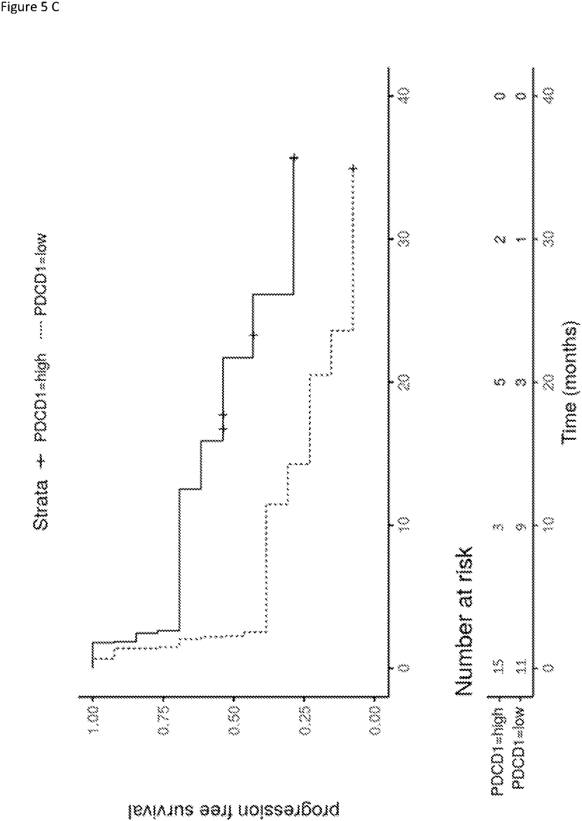

To illustrate how well these DEGs discriminate between patients with and without clinical benefit, PFS curves were generated. Patients were dichotomized according to the classification output of the model including 5 DNA replication gene panel (DLGAP5, TOP2A, CDCA2, E2F8 and SMC1A), one of the IFN panel (PDCD1), or their combination (FIGS. 5). Six-month PFS was better in patients stratified with the DNA replication gene panel (83.3% versus 28.6%,

FIG. 5A), confirming what found performance analysis. The difference in PFS were less pronounced when PDCD1 was added to the DNA replication panel or when it was used alone (FIGS. 5B and C).

### REFERENCES

1. Fradet, Y.; Bellmunt, J.; Vaughn, D.J.; Lee, J.L.; Fong, L.; Vogelzang, N.J.; Climent, M.A.; Petrylak, D.P.; Choueiri, T.K.; Necchi, A; et al. Randomized phase III KEYNOTE-045 trial of pembrolizumab versus paclitaxel, docetaxel, or vinflunine in recurrent advanced urothelial cancer: results of >2 years of follow-up. Ann. Oncol. 2019, 30, 970-976, doi:10.1093/annonc/mdz127.

2. Powles, T.; Durán, I.; van der Heijden, M.S.; Loriot, Y.; Vogelzang, N.J.; De Giorgi, U.; Oudard, S.; Retz, M.M.; Castellano, D.; Bamias, A.; et al. Atezolizumab versus chemotherapy in patients with platinum-treated locally advanced or metastatic urothelial carcinoma (IMvigor211): a multicentre, open-label, phase 3 randomised controlled trial. Lancet 2018, 391, 748-757, doi:10.1016/S0140-6736(17)33297-X.

3. Powles, T.; Kockx, M.; Rodriguez-Vida, A.; Duran, I.; Crabb, S.J.; Van Der Heijden, M.S.; Szabados, B.; Pous, A.F.; Gravis, G.; Herranz, U.A.; et al. Clinical efficacy and biomarker analysis of neoadjuvant atezolizumab in operable urothelial carcinoma in the ABACUS trial. Nat. Med. 2019, 25, 1706-1714, doi:10.1038/s41591-019-0628-7.

4. Necchi, A.; Anichini, A.; Raggi, D.; Briganti, A.; Massa, S.; Lucianò, R.; Colecchia, M.; Giannatempo, P.; Mortarini, R.; Bianchi, M.; et al. Pembrolizumab as Neoadjuvant Therapy Before Radical Cystectomy in Patients With Muscle-Invasive Urothelial Bladder Carcinoma (PURE-01): An Open-Label, Single-Arm, Phase II Study. J. Clin. Oncol. 2018, 36, 3353-3360, doi: 10.1200/JCO.18.01148.

5. Samstein, R.M.; Lee, C.H.; Shoushtari, A.N.; Hellmann, M.D.; Shen, R.; Janjigian, Y.Y.; Barron, D.A.; Zehir, A.; Jordan, E.J.; Omuro, A.; et al. Tumor mutational load predicts survival after immunotherapy across multiple cancer types. Nat. Genet. 2019, 51, 202-206.

6. Rui, X.; Gu, T.-T.; Pan, H.-F.; Zhang, H.-Z. Evaluation of PD-L1 biomarker for immune checkpoint inhibitor (PD-⅟PD-L1 inhibitors) treatments for urothelial carcinoma patients: A meta-analysis. Int. Immunopharmacol. 2019, 67, 378-385, doi:10.1016/J.INTIMP.2018.12.018.

7. Keegan, A.; Ricciuti, B.; Garden, P.; Cohen, L.; Nishihara, R.; Adeni, A.; Paweletz, C.; Supplee, J.; Jänne, P.A.; Severgnini, M.; et al. Plasma IL-6 changes correlate to PD-1 inhibitor responses in NSCLC. J. Immunother. Cancer 2020, 8, doi:10.1136/jitc-2020-000678.

8. Sanmamed, M.F.; Perez-Gracia, J.L.; Schalper, K.A.; Fusco, J.P.; Gonzalez, A.; Rodriguez-Ruiz, M.E.; Oñate, C.; Perez, G.; Alfaro, C.; Martin-Algarra, S.; et al. Changes in serum interleukin-8 (IL-8) levels reflect and predict response to anti-PD-1 treatment in melanoma and non-small-cell lung cancer patients. Ann. Oncol. 2017, 28, 1988-1995, doi:10.1093/annonc/mdx190.

9. Simon, S.; Voillet, V.; Vignard, V.; Wu, Z.; Dabrowski, C.; Jouand, N.; Beauvais, T.; Khammari, A.; Braudeau, C.; Josien, R.; et al. PD-1 and TIGIT coexpression identifies a circulating CD8 T cell subset predictive of response to anti-PD-1 therapy. J. Immunother. Cancer 2020, 8, doi:10.1136/jitc-2020-001631.

10. Huang, A.C.; Postow, M.A.; Orlowski, R.J.; Mick, R.; Bengsch, B.; Manne, S.; Xu, W.; Harmon, S.; Giles, J.R.; Wenz, B.; et al. T-cell invigoration to tumour burden ratio associated with anti-PD-1 response. Nature 2017, 545, 60-65, doi:10.1038/nature22079.

11. Kamphorst, A.O.; Pillai, R.N.; Yang, S.; Nasti, T.H.; Akondy, R.S.; Wieland, A.; Sica, G.L.; Yu, K.; Koenig, L.; Patel, N.T.; et al. Proliferation of PD-1+ CD8 T cells in peripheral blood after PD-1-targeted therapy in lung cancer patients. Proc. Natl. Acad. Sci. U S. A. 2017, 114, 4993-4998, doi:10.1073/pnas.1705327114.

1. A method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and,
at least one gene selected among those listed in Table 1,
wherein the at least one ECM gene cluster comprises COL14A1 and the at least one gene of Table 1 comprises MORN4A, and
wherein differential transcription and/or expression and/or activity level of the gene panel, in the biological sample, relative to the level of corresponding transcription and/or expression and/or activity level of the gene panel determined previously, is predictive of the patient’s response to said treatment.

2. The method of claim 1, wherein the gene panel further comprises at least one gene selected among the cAMP cluster (Table 3).

3. The method of claim 2, wherein the least one gene selected among the cAMP cluster (Table 3) is selected from the group comprising PDE10A, CASR, and KCNJ6, or a combination of two or more thereof.

4. The method of claim 2, wherein the least one gene selected among the cAMP cluster (Table 3) consists of PDE10A, CASR, and KCNJ6.

5. The method of any one of the preceding claims, wherein the least one gene selected among those listed in Table 1 is further selected from the group comprising BNIPL, CCDC40, and DHRS2, or a combination of two or more thereof.

6. The method of any one of the preceding claims, wherein the least one gene selected among those listed in Table 1 consists of MORN4A, BNIPL, CCDC40, and DHRS2.

7. The method of any one of the preceding claims, wherein the least one gene selected the Extra Cellular Matrix (ECM) cluster (Table 2) is further selected from the group comprising DOCK1, and ADAMTS2, or a combination thereof.

8. The method of any one of the preceding claims, wherein the disease is a cancer or an autoimmune disease.

9. The method of claim 8, wherein the cancer is selected from the group comprising urothelial cancer, urinary bladder cancer, lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, hepatic cancer, brain cancer and skin cancer, or a combination of one or more thereof.

10. The method of claim 9, wherein the urinary bladder cancer is urothelial cancer, preferably metastatic urothelial cancer.

11. The method of any one of the preceding claims, wherein the treatment based on ICBT is selected among the group comprising a PD-1 inhibitor, a PD-L1 inhibitor and a CTLA-4 inhibitor, or combination of one or more thereof.

12. The method of any one of the preceding claims, wherein the treatment based on ICBT comprises treatment with monoclonal antibodies (mAbs) specific to PD-1, PD-L1 or CTLA-4, or combination of one or more thereof.

13. The method of any one of the preceding claims, wherein the differential transcription and/or expression and/or activity level of the gene panel corresponds to a differential expression of the transcripts of the genes of the panel.

14. The method of any one of the preceding claims, wherein the differential transcription and/or expression and/or activity level of the gene panel corresponds to a downregulated or upregulated expression of said genes.

15. The method of claim 14, wherein the differential transcription and/or expression and/or activity level of the gene panel corresponds to a downregulated expression of said genes.

16. The method of claim 15, wherein the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously.

17. The method of any one of the preceding claims, wherein the level of transcription and/or expression of a gene panel is performed by whole transcriptome RNA sequencing or targeted RNA seq .

18. The method of any one of the preceding claims, wherein, if the patient having a predetermined disease is predicted to respond to said treatment, the treatment is started.

19. The method of any one of claims 1 to 17, wherein, if the patient having a predetermined disease is predicted not to respond to said treatment, the method further comprises a step of adapting the treatment.

20. The method of claim 19, wherein the step of adapting the treatment comprises not administering the envisioned treatment or inhibitor and/or adapting the dose of the inhibitor.

21. The method of any one of the preceding claims, wherein the level of corresponding transcription and/or expression and/or activity level of the gene panel determined previously has been determined before starting the ICBT.

22. A method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Extra Cellular Matrix (ECM) cluster (Table 2) and
wherein differential transcription and/or expression and/or activity level of the gene panel, in the biological sample, relative to the level of corresponding transcription and/or expression and/or activity level of the gene panel determined previously, is predictive of the patient’s response to said treatment.

23. A method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among those listed in Table 1, and
wherein differential transcription and/or expression and/or activity level of the gene panel, in the biological sample, relative to the level of corresponding transcription and/or expression and/or activity level of the gene panel determined previously, is predictive of the patient’s response to said treatment.

24. A method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the cAMP cluster (Table 3),
wherein differential transcription and/or expression and/or activity level of the gene panel, in the biological sample, relative to the level of corresponding transcription and/or expression and/or activity level of the gene panel determined previously, is predictive of the patient’s response to said treatment.

25. A method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the DNA replication cluster of Table 4,
wherein differential transcription and/or expression and/or activity level of the gene panel, in the biological sample, relative to the level of corresponding transcription and/or expression and/or activity level of the gene panel determined previously, is predicting that the patient is responsive to said treatment.

26. A method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the Interferon cluster genes (Table 5),
wherein differential transcription and/or expression and/or activity level of the gene panel, in the biological sample, relative to the level of corresponding transcription and/or expression and/or activity level of the gene panel determined previously, is predicting that the patient is responsive to said treatment.

27. The method according to any one of the preceding claims wherein the biological sample is selected from the group comprising whole blood, serum, plasma, semen, saliva, tears, urine, fecal material, sweat, buccal smears, skin, tumor tissue and cancer cells, or a combination of one or more of thereof.

28. The method according to any one of the preceding claims wherein the level of transcription and/or expression and/or activity of a gene panel is expressed as a score.

29. A method for determining if a patient having a predetermined disease is responsive to a treatment based on immune checkpoint blockade therapy or treatment (ICBT), said method comprising detecting in a biological sample obtained from said patient the level of transcription and/or expression and/or activity of a gene panel comprising:
at least one gene selected among the DNA replication cluster (Table 4) and,
at least one gene selected among the gene interferon cluster (Table 5),
wherein the at least one DNA replication gene cluster comprises PLK4 and the at least one interferon cluster gene comprises PDCD1, and
wherein differential transcription and/or expression and/or activity level of the gene panel, in the biological sample, relative to the level of corresponding transcription and/or expression and/or activity level of the gene panel determined previously, is indicative of whether the patient responds or not to said treatment.

30. The method of claim 29, wherein the least one gene selected among the DNA replication cluster is further selected from the group further comprising CENPE, CDCA2, E2F8, TOP2A, DLGAP5, SGOL2, NOTCH3, CCNB2, ASPM, SMC1A and MCM10, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, or preferably a combination of eleven thereof.

31. The method of any one of claims 29 to 30, wherein the least one gene selected among the gene interferon cluster is selected from the group further comprising CLC, NMI, FCGR1A, FCGR1B, BCL2L14, IFITM3, STAT1, GBP2, C5, IFIT5, IFI35, TRIM22, IL10, and IDO1, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of twelve or more thereof, preferably a combination of thirteen or more thereof, or preferably a combination of fourteen thereof.

32. The method of any one of claims 29 to 31, wherein the gene panel further comprises at least one gene selected among those listed in Table 6, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of fifteen or more thereof, preferably a combination of twenty or more thereof, preferably a combination of twenty five or more thereof, preferably a combination of thirty or more thereof, preferably a combination of thirty five or more thereof, preferably a combination of forty or more thereof, or preferably a combination of forty-two thereof.

33. The method of any one of claims 29 to 32, wherein the disease is a cancer or an autoimmune disease.

34. The method of claim 33, wherein the cancer is selected from the group comprising urothelial cancer, lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer, brain cancer and skin cancer, or a combination of one or more thereof.

35. The method of claim 34, wherein the urinary bladder cancer is urothelial cancer, preferably metastatic urothelial cancer.

36. The method of any one of claims 29 to 35, wherein the treatment based on ICBT is selected among the group comprising a PD-1 inhibitor, a PD-L1 inhibitor and a CTLA-4 inhibitor, or combination of one or more thereof.

37. The method of any one of claims 29 to 36, wherein the treatment based on ICBT comprises treatment with monoclonal antibodies (mAbs) specific to PD-1, PD-L1 or CTLA-4, or combination of one or more thereof.

38. The method of any one of claims 29 to 37, wherein the differential transcription and/or expression and/or activity level of the gene panel corresponds to a differential expression of the transcripts of the genes of the panel.

39. The method of any one of claims 29 to 38, wherein the differential transcription and/or expression and/or activity level of the gene panel corresponds to a downregulated or upregulated expression of said genes.

40. The method of claim 39, wherein the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000 (% when compared to the level of corresponding transcription and/or expression and/or activity level of the gene panel determined previously.

41. The method of claim 39, wherein the upregulated differential transcription and/or expression and/or activity of said gene panel corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously.

42. The method of any one of the claims 29 to 41, wherein the level of transcription and/or expression of a gene panel is performed by whole transcriptome RNA sequencing or targeted RNA seq.

43. The method of any one of claims 29 to 42, wherein, if the patient having a predetermined disease is determined as responsive to said treatment, the treatment is continued.

44. The method of any one of claims 29 to 43, wherein, if the patient having a predetermined disease is determined as not responsive to said treatment, the method further comprises a step of adapting the treatment.

45. The method of claim 44, wherein the step of adapting the treatment comprises changing the treatment for another treatment or inhibitor and/or adapting the dose of the inhibitor.

46. The method of any one of claims 29 to 45, wherein the level of corresponding transcription and/or expression and/or activity level of the gene panel determined previously has been determined before starting the ICBT.

47. A computer-implemented method for implementing a method for predicting if a patient having a predetermined disease will respond to a treatment based on immune checkpoint blockade therapy or treatment (ICBT) of any one of claims 1 to 28, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined previously, whereby differential transcription and/or expression and/or activity level of the gene panel, in the biological sample, relative to the level of corresponding transcription and/or expression and/or activity level of the gene panel determined previously, is predictive of the patient’s response to said treatment.

48. A computer-implemented method for implementing a method for determining if a patient having a predetermined disease is responsive to a treatment based on immune checkpoint blockade therapy or treatment (ICBT) of any one of claims 29 to 46, said computer-implemented method comprising
i) scoring the level of transcription and/or expression and/or activity of a gene panel in the biological sample of the patient,
ii) comparing the determined score to the level of corresponding transcription and/or expression and/or activity level of the gene panel determined in a control biological sample, whereby wherein differential transcription and/or expression and/or activity level of the gene panel, in the biological sample of the patient, relative to the level of corresponding transcription and/or expression and/or activity level of the gene panel determined previously, is indicative of whether the patient will respond or not to said treatment.

49. A method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising:
at least one gene selected among the list of Table 6,
wherein differential transcription and/or expression and/or activity level of the gene panel, in the biological sample, relative to the level of corresponding transcription and/or expression and/or activity level of the gene panel determined previously, is predicting that the patient is responsive to said treatment.

50. Use of a gene panel comprising at least one gene selected among the Extra Cellular Matrix (ECM) cluster (Table 2) and, at least one gene selected among those listed in Table 1, wherein the at least one ECM gene cluster comprises COL14A1 and the at least one gene of Table 1 comprises MORN4A, for predicting if a patient having a predetermined disease will respond to a treatment based on immune checkpoint blockade therapy or treatment (ICBT).

51. The use of claim 50, further comprises at least one gene selected among the cAMP cluster (Table 3).

52. The use of claim 51, wherein the least one gene selected among the cAMP cluster (Table 3) is selected from the group comprising PDE10A, CASR, and KCNJ6, or a combination of two or more thereof.

53. The use of claim 51, wherein the least one gene selected among the cAMP cluster (Table 3) consists of PDE10A, CASR, and KCNJ6.

54. The use of any one of claims 50 to 53, wherein the least one gene selected among those listed in Table 1 is further selected from the group comprising BNIPL, CCDC40, and DHRS2, or a combination of two or more thereof.

55. The use of any one of claims 50 to 54, wherein the least one gene selected among those listed in Table 1 consists of MORN4A, BNIPL, CCDC40, and DHRS2.

56. The use panel of any one of claims 50 to 55, wherein the least one gene selected the Extra Cellular Matrix (ECM) cluster (Table 2) is further selected from the group comprising DOCK1, and ADAMTS2, or a combination thereof.

57. Use of a gene panel comprising at least one gene selected among the DNA replication cluster (Table 4) and, at least one gene selected among the gene interferon cluster (Table 5), wherein the at least one DNA replication gene cluster comprises PLK4 and the at least one interferon cluster gene comprises PDCD1, for determining if a patient having a predetermined disease is responsive to a treatment based on immune checkpoint blockade therapy or treatment (ICBT).

58. The use of claim 57, wherein the least one gene selected among the DNA replication cluster is further selected from the group further comprising CENPE, CDCA2, E2F8, TOP2A, DLGAP5, SGOL2, NOTCH3, CCNB2, ASPM, SMC1A and MCM10, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination often or more thereof, or preferably a combination of eleven thereof.

59. The use of any one of claims 57 to 58, wherein the least one gene selected among the gene interferon cluster is selected from the group further comprising CLC, NMI, FCGR1A, FCGR1B, BCL2L14, IFITM3, STAT1, GBP2, C5, IFIT5, IFI35, TRIM22, IL10, and IDO1, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of eleven or more thereof, preferably a combination of twelve or more thereof, preferably a combination of thirteen or more thereof, or preferably a combination of fourteen thereof.

60. The use of any one of claims 57 to 59, wherein the gene panel further comprises at least one gene selected among those listed in Table 6, or a combination of two or more thereof, preferably a combination of three or more thereof, preferably a combination of four or more thereof, preferably a combination of five or more thereof, preferably a combination of six or more thereof, preferably a combination of seven or more thereof, preferably a combination of eight or more thereof, preferably a combination of nine or more thereof, preferably a combination of ten or more thereof, preferably a combination of fifteen or more thereof, preferably a combination of twenty or more thereof, preferably a combination of twenty five or more thereof, preferably a combination of thirty or more thereof, preferably a combination of thirty five or more thereof, preferably a combination of forty or more thereof, or preferably a combination of forty-two thereof.

61. Use of a gene panel for predicting if a patient having a predetermined disease will respond to a treatment based on immune checkpoint blockade therapy or treatment (ICBT), comprising:
at least one gene selected among the Extra Cellular Matrix (ECM) cluster (Table 2) and/or
at least one gene selected among those listed in Table 1, and/or
at least one gene selected among the cAMP cluster (Table 3), and/or
at least one gene selected among the DNA replication cluster of Table 4.

62. Use of a gene panel for determining if a patient having a predetermined disease is responsive to a treatment based on immune checkpoint blockade therapy or treatment (ICBT), comprising:
at least one gene selected among the DNA replication cluster of Table 4, and/or
at least one gene selected among the Interferon cluster genes (Table 5), and/or
at least one gene selected among those listed in Table 6.

63. A kit for performing a method according to any one of claims 1 to 46 or 49, said kit comprising
a) means and/or reagents for determining the level of transcription and/or expression and/or activity of said gene panel in a biological sample from said patient, and
b) instructions for use.

64. A method of treatment of a cancer or an autoimmune disease, comprising
i) detecting in a biological sample obtained from said patient the level of transcription and/or expression and/or activity of a gene panel of any one of claims 1 to 24,
ii) and treating the patient based upon whether a differential transcription and/or expression and/or activity level of said gene panel, in the biological sample, relative to the level of corresponding transcription and/or expression and/or activity level of the gene panel determined previously, is predictive of the patient’s response to said treatment.

65. A method of treatment of a cancer or an autoimmune disease, comprising
i) detecting in a biological sample obtained from said patient the level of transcription and/or expression and/or activity of a gene panel of any one of claims 25 to 46 or 49,
ii) and treating the patient based upon whether a differential transcription and/or expression and/or activity level of said gene panel, in the biological sample, relative to the level of corresponding transcription and/or expression and/or activity level of the gene panel determined previously, is predicting that the patient is responsive to said treatment.

66. The method of treatment of claim 64 or 65, wherein the cancer is mUC.
